(19) United States
(12) Patent Application Publication
(10) Pub. No.: US 2024/0390652 A1
(43) Pub. Date: Nov. 28, 2024

(54) Catheter Placement Device Including a Mechanical Advantage Mechanism

(71) Applicant: Bard Access Systems, Inc., Salt Lake City, UT (US)

(72) Inventor: Daniel B. Blanchard, Bountiful, UT (US)

(21) Appl. No.: 18/202,702

(22) Filed: May 26, 2023

(51) Int. Cl. A61M 25/06; A61M 25/00

(52) U.S. Cl. CPC A61M 25/0606; A61M 25/0097

(57) ABSTRACT

An insertion tool for inserting a catheter into a patient's body is disclosed. The insertion tool unifies needle insertion, guidewire advancement, and catheter insertion in a single device. In one embodiment, the insertion tool comprises a housing in which at least a portion of the catheter is initially disposed, a hollow needle distally extending from the housing with at least a portion of the catheter pre-disposed over the needle, and a guidewire pre-disposed within the needle. A guidewire advancement assembly is also included for selectively advancing the guidewire distally past a distal end of the needle in preparation for distal advancement of the catheter. A catheter advancement assembly is also included for selectively advancing the catheter into the patient. The catheter advancement assembly includes a mechanical advantage mechanism coupled between the slide of the insertion tool and the catheter.

### BRIEF SUMMARY

Disclosed herein is a catheter insertion device assembly that, according to some embodiments, includes a catheter having a catheter tube defining a catheter lumen extending between a catheter distal end and a catheter hub at a catheter proximal end, where the catheter hub disposed within a housing. The assembly further includes a needle configured for insertion into a patient between a skin surface and a blood vessel, where the needle defines a needle lumen extending between a needle distal end and a needle proximal end coupled with the housing, and where the needle is pre-disposed within the catheter lumen such that the needle distal end extends beyond the catheter distal end and the needle proximal end extends proximally beyond the catheter hub. The assembly further includes a guidewire extending between a guidewire distal end and a guidewire proximal portion, where the guidewire is pre-disposed within the needle lumen such that the guidewire distal end is positioned proximal the needle distal end and the guidewire proximal portion extends proximally beyond the needle proximal end. The assembly further includes a slide displaceable along an exterior of the housing, where the slide is coupled with the guidewire proximal portion such that a displacement of the slide causes a displacement of the guidewire. The assembly further includes a mechanical advantage mechanism coupled between the slide and the catheter hub such that the slide provides an input force to the mechanical advantage mechanism and the mechanical advantage mechanism provides an output force to the catheter hub in response to the input force, where the output force is greater than the input force.

In some embodiments, the input force and the output force are each distally oriented, and in some embodiments, the output force is greater than the input force by a factor of two.

In some embodiments, the displacement of the slide causes a displacement of the catheter and a simultaneous displacement of the guidewire, and in some embodiments, the displacement of the catheter is less than the simultaneous displacement of the guidewire.

In some embodiments, the mechanical advantage mechanism includes a lever, and in some embodiments, the lever includes an opening, where the needle passes through the opening.

In some embodiments, the lever includes (i) a first end coupled with the slide, (ii) a second end defining a fulcrum with a bottom housing portion, and (iii) an intermediate point coupled with the catheter hub. In such embodiments, a distal displacement of the slide with respect to the bottom housing portion causes a distal displacement of the catheter with respect to the bottom housing portion.

In some embodiments, the lever includes (i) a first end coupled with the slide, (ii) a second end defining a fulcrum with a hub of the needle, and (iii) an intermediate point coupled with the catheter hub. In such embodiments, a distal displacement of the slide with respect to the bottom housing portion causes a distal displacement of the catheter with respect to the needle.

In some embodiments, the lever includes (i) a first end coupled with the slide, (ii) a second end coupled with a hub of the needle, and (iii) an intermediate point defining a fulcrum with the catheter hub. In such embodiments, a distal displacement of the slide with respect to the bottom housing portion causes a distal displacement of the catheter with respect to the needle.

In some embodiments, the lever includes (i) a first end slidably coupled with a cam surface of the slide, (ii) a second end coupled with the catheter hub, and (iii) intermediate point adjacent a bend of the lever defining a fulcrum with a hub of the needle. In such embodiments, a distal displacement of the slide with respect to the bottom housing portion causes a transverse displacement of the first end which in turn causes a distal displacement of the catheter with respect to the needle.

In some embodiments, the mechanical advantage mechanism includes a tension member having (i) a first end coupled with the slide, (ii) a second end coupled with a bottom housing portion, and (iii) a loop portion coupled with the catheter hub. In such embodiments, a distal displacement of the slide with respect to the bottom housing portion causes the loop portion to distally displace the catheter with respect to the bottom housing portion.

In some embodiments, the mechanical advantage mechanism includes (i) a first gear rack coupled with the slide, (ii) a second gear rack coupled with a bottom housing portion, and (iii) a pinion gear coupled with the catheter hub, where the pinion gear is in mesh with the first gear rack and the second gear rack. In such embodiments, a distal displacement of the slide with respect to the bottom housing portion causes the pinion gear to rotate and distally displace along the bottom housing portion resulting in distal co-displacement of the catheter with the pinion gear with respect to the bottom housing portion.

In some embodiments, the assembly further includes a safety assembly configured to cover a distal tip of the needle upon withdrawal of the needle from the catheter, where the safety assembly is coupled between the catheter hub and the mechanical advantage mechanism. In such embodiments, distal displacement of the slide causes distal displacement of the safety assembly which in turn causes distal displacement of the catheter.

In some embodiments, the slide is configured to displace a first distance and a subsequent second distance, where displacement of the slide the first distance causes the guidewire to distally displace a first guidewire distance with respect to the needle, and where displacement of the slide the subsequent second distance causes (i) the guidewire to distally displace a second guidewire distance with respect to the needle and (ii) the catheter to displace a first catheter distance with respect to the needle, where the first catheter distance less than the second guidewire distance.

Also disclosed herein is a method of placing a catheter within a blood vessel that, according to some embodiments, includes inserting a needle of a catheter insertion device assembly through a skin of a patient such at a distal end of the needle is disposed within the blood vessel, where (i) the needle is pre-disposed within a lumen of a catheter of the catheter insertion device assembly, and (ii) a guidewire of the catheter insertion device assembly is pre-disposed with a lumen of the needle. The method further includes distally advancing the guidewire along the lumen of the needle such that the guidewire extends beyond the distal end of the needle and distally advancing the catheter a catheter distance along the needle such that a distal end of the catheter is displaced from a position proximal the distal end the needle to a position distal the distal end the needle, where distally advancing the catheter includes distally displacing a slide of the catheter insertion device assembly a slide distance with respect to a housing of the catheter insertion device assembly, and where the slide distance is greater than the catheter distance.

In some embodiments of the method, distally displacing the slide includes exerting a distally oriented first force on the slide, and distally advancing the catheter includes exerting a distally oriented second force on the catheter, where the second force is greater than the first force.

In some embodiments of the method, distally displacing the slide includes rotating a lever about a fulcrum, where the fulcrum coupled with a hub of the catheter, a hub of the needle, or the housing.

In some embodiments of the method, the catheter insertion device includes a tension member having (i) a first end coupled with the slide, (ii) a second end coupled with the housing, and (iii) a loop portion coupled with the catheter. In such embodiments, a distal displacement of the slide with respect to the housing causes the loop portion to distally displace the catheter along the needle.

In some embodiments of the method, the catheter insertion device includes (i) a first gear rack coupled with the slide, (ii) a second gear rack coupled with the housing, and (iii) a pinion gear coupled with the catheter, where the pinion gear in mesh with the first gear rack and the second gear rack. In such embodiments, a distal displacement of the slide with respect to the housing causes the pinion gear to rotate and distally displace along the housing thereby causing the catheter to distally displace along the needle.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A and 1B are various views of a catheter insertion device according to one embodiment;

FIGS. 2A and 2B are various exploded views of the catheter insertion device of FIGS. 1A and 1B;

FIGS. 3A and 3B show various views of one stage of use of the catheter insertion device of FIGS. 1A and 1B according to one embodiment;

FIGS. 4A and 4B show various views of one stage of use of the catheter insertion device of FIGS. 1A and 1B according to one embodiment;

FIGS. 5A and 5B show various views of one stage of use of the catheter insertion device of FIGS. 1A and 1B according to one embodiment;

FIGS. 6A and 6B show various views of one stage of use of the catheter insertion device of FIGS. 1A and 1B according to one embodiment;

FIGS. 7A and 7B show various views of one stage of use of the catheter insertion device of FIGS. 1A and 1B according to one embodiment;

FIG. 8 shows one stage of use of the catheter insertion device of FIGS. 1A and 1B according to one embodiment;

FIG. 9 shows one stage of use of the catheter insertion device of FIGS. 1A and 1B according to one embodiment;

FIGS. 10A-10C shows various views of a needle safety component and environment for a catheter insertion device, according to one embodiment;

FIGS. 11A-11D are various views of a catheter insertion device according to one embodiment;

FIGS. 12A and 12B are various views of a portion of the catheter insertion device of FIGS. 11A-11D;

FIGS. 13A and 13B are various views of a portion of the catheter insertion device of FIGS. 11A-11D;

FIGS. 14A-14F show various stages of use of the catheter insertion device of FIGS. 11A-11D according to one embodiment;

FIG. 15 is an exploded view of a catheter insertion device according to one embodiment;

FIG. 16 is a perspective view of a portion of a guidewire lever according to one embodiment;

FIGS. 17A and 17B are cutaway views of a proximal portion of the catheter insertion device of FIG. 15;

FIG. 18 is a perspective view of a proximal portion of the top housing portion of the catheter insertion device of FIG. 15;

FIG. 19 is a cutaway view of a proximal portion of the catheter insertion device of FIG. 15;

FIGS. 20A and 20B are various views of a needle safety component according to one embodiment;

FIGS. 21A-21D are various views of the needle safety component of FIGS. 20A and 20B and an accompanying carriage;

FIGS. 22A and 22B are cutaway views of a proximal portion of the catheter insertion device of FIG. 15 according to one embodiment;

FIGS. 23A-23D are cross-sectional side views of a portion of the device of FIG. 15 illustrating various embodiments of a mechanical advantage mechanism having a lever according to some embodiments;

FIG. 23E is cross-sectional side view of a portion of the device of FIG. 15 illustrating another embodiment of a mechanical advantage mechanism having a tension member according to some embodiments;

FIG. 23F is cross-sectional side view of a portion of the device of FIG. 15 illustrating another embodiment of a mechanical advantage mechanism having a pinion gear disposed between opposing gear racks according to some embodiments; and

FIG. 24 is block diagram of a method of placing a catheter within a blood vessel according to some embodiments.

### DETAILED DESCRIPTION

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

Embodiments of the present invention are generally directed to a tool for assisting with the placement into a patient of a catheter or other tubular medical device. For example, catheters of various lengths are typically placed into a body of a patient so as to establish access to the patient's vasculature and enable the infusion of medicaments or aspiration of body fluids. The catheter insertion tool to be described herein facilitates such catheter placement. Note that, while the discussion below focuses on the placement of catheters of a particular type and relatively short length, catheters of a variety of types, sizes, and lengths can be inserted via the present device, including peripheral IV's intermediate or extended-dwell catheters, PICC's, central venous catheters, etc. In one embodiment, catheters having a length between about 2.5 inches and about 4.5 inches can be placed, though many other lengths are also possible. In another embodiment a catheter having a length of about 3.25 inches can be placed.

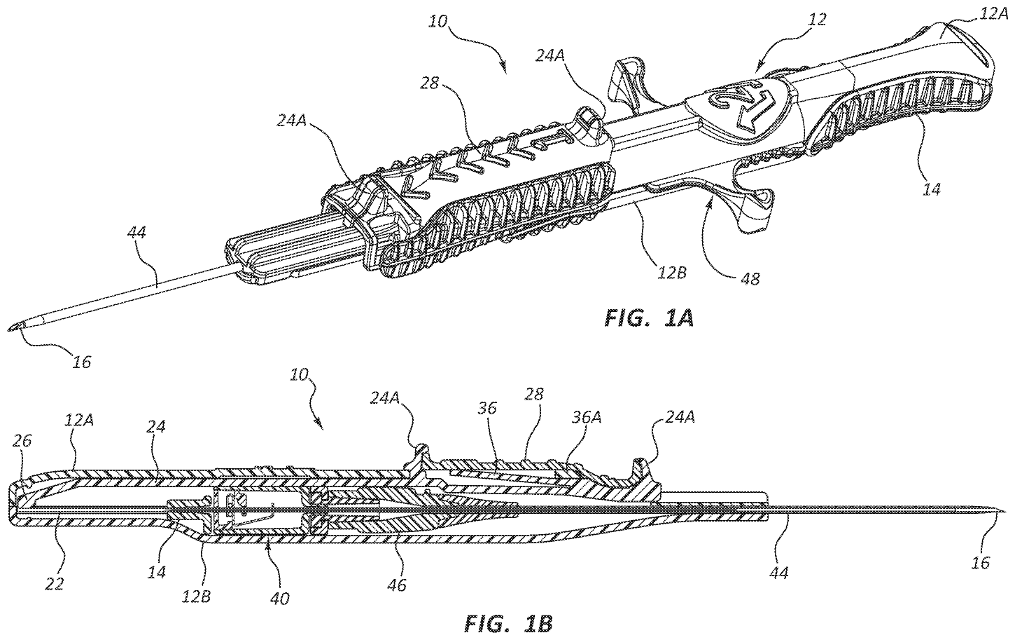

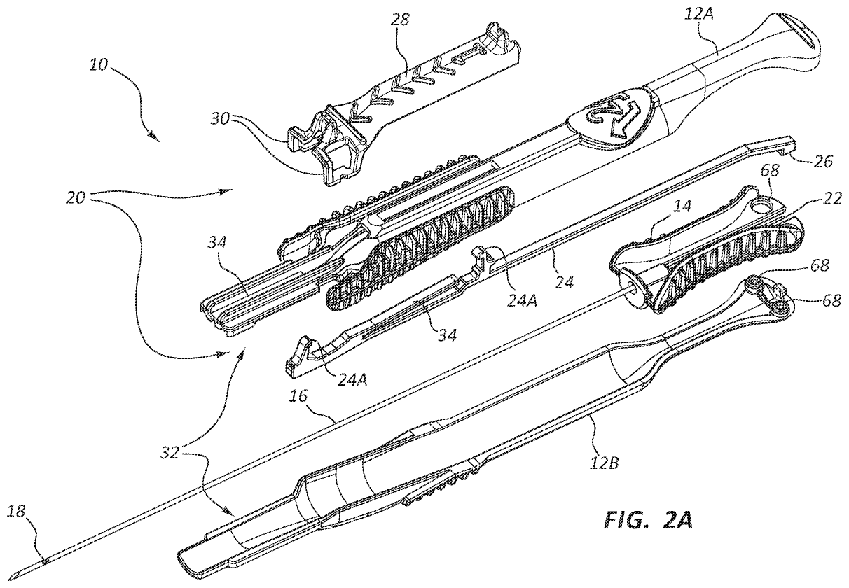

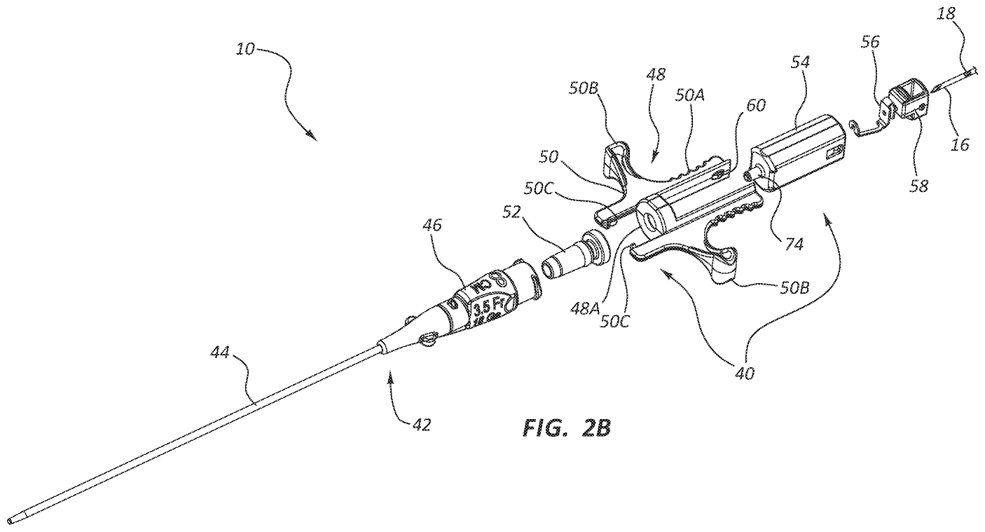

Reference is first made to FIGS. 1A-1B and 2A-2B, which depict various details regarding a catheter insertion tool (“insertion tool”), generally depicted at 10, according to one embodiment. As shown, the insertion tool 10 includes a housing 12 that in turn includes a top housing portion 12A separably mated with a bottom housing portion 12B. A needle hub 14 supporting a hollow needle 16 is interposed between the housing portions 12A and 12B. The needle 16 extends distally from the needle hub 14 so as to extend through the body of the insertion tool 10 and out a distal end of the housing 12. In another embodiment, the needle is at least partially hollow while still enabling the functionality described herein.

A notch 18 is defined through the wall of the needle 16 proximate the distal end thereof. The notch 18 enables flashback of blood to exit the lumen defined by the hollow needle 16 once access to the patient's vasculature is achieved during catheter insertion procedures. Thus, blood exiting the notch 18 can be viewed by a clinician to confirm proper needle placement in the vasculature, as will be explained further below.

The insertion tool 10 further includes a guidewire advancement assembly 20 for advancing a guidewire 22 through the needle 16 and into the vasculature of the patient once access by the needle has been achieved. The guidewire 22 is pre-disposed within the lumen of the needle 16, with a proximal end of the guidewire positioned proximate the proximal end of the needle hub 14, as best seen in FIGS. 1B and 2A. The guidewire advancement assembly 20 includes a guidewire lever 24 that selectively advances the guidewire in a distal direction during use of the insertion tool 10 such that the distal portion of the guidewire extends beyond the distal end of the needle 16. The guidewire lever 24 includes a lever tab 26 that engages the proximal end of the guidewire 22 so to push the guidewire through the lumen of the needle 16.

The guidewire advancement assembly 20 further includes a slide 28 that is slidably attached to the top housing portion 12A. Two tabs 24A of the guidewire lever 24 operably attach to the slide 28 so that selective movement by a user of the slide results in corresponding movement of the lever 24, and by extension, the guidewire 22. Engagement of the lever tabs 24A to the slide 28 also maintains attachment of the slide to the housing 12. Of course, other engagement schemes to translate user input to guidewire movement could also be employed. Suitable tracks are included in the top housing portion 12A to enable sliding movement of the slide 28 and the lever 24, including a track 34 extending to the distal end of the housing 12.

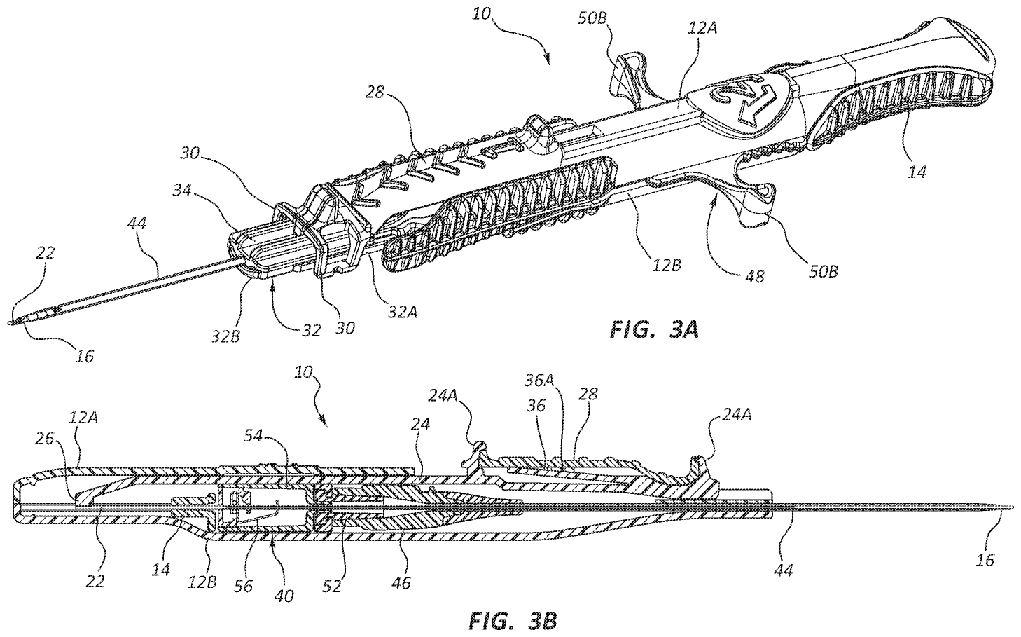

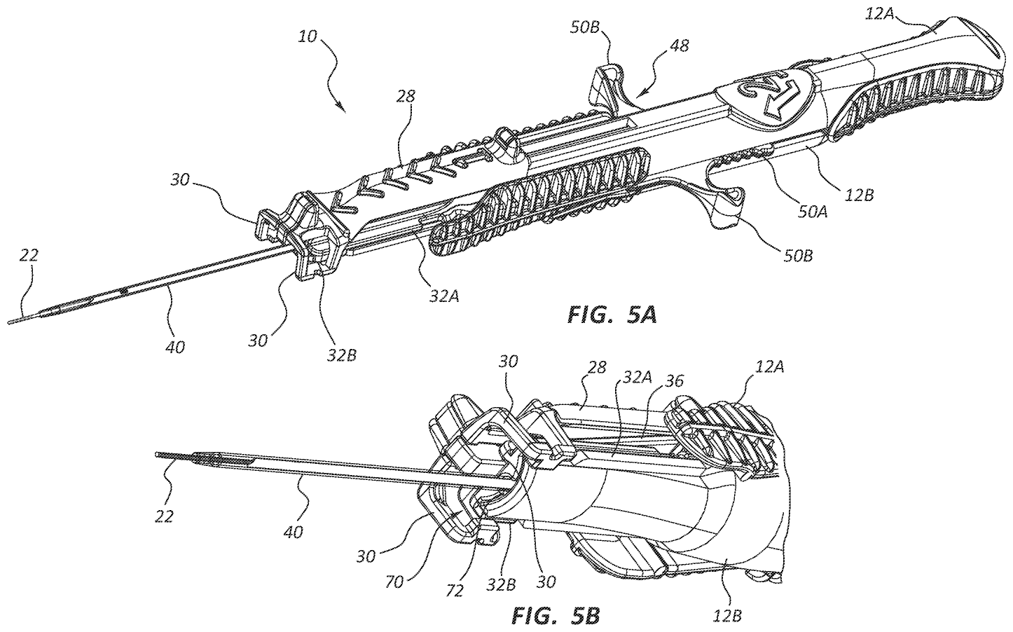

The slide 28 includes two arms 30 that wrap partially about rails 32 defined by the housing 12. In particular, during initial distal advancement of the slide 28, the arms 30 slide on a bottom housing rail 32A, best seen in FIG. 5B. During further distal advancement of the slide 28, the arms 30 slide past the bottom housing rail 32A and on to a top housing rail 32B, best seen in FIGS. 2A and 3A. With the arms 30 of the slide 28 no longer engaged with the bottom housing rail 32A, the two housing portions 12A and 12B are able to separate, as will be described further below.

The guidewire lever 24 includes a locking arm 36 resiliently disposed so as to spring up and engage an extension 36A defined in the interior of the top housing portion 12A when the slide 28 has been fully slid distally. This prevents inadvertent retraction of the guidewire 22 once distally extended, which could otherwise cause unintended severing of a distal portion of the guidewire by the distal tip of the needle 16 during insertion procedures. Note that engagement of the locking arm 36 with the extension 36A can provide tactile and/or audible feedback to the user in one embodiment so as to indicate full distal extension of the guidewire 22.

The insertion tool 10 further includes a catheter advancement assembly 40 for selectively advancing in a distal direction a catheter 42, pre-disposed in the housing 12, and including a catheter tube 44 and a hub 46 at a proximal end thereof. As seen in FIGS. 1A and 1B, the catheter 42 is partially and initially pre-disposed within a volume defined by the housing 12 such that the lumen of the catheter tube 44 is disposed over the needle 16, which in turn is disposed over the guidewire 22, as mentioned.

In particular, the catheter advancement assembly 40 includes a handle 48 that defines a base 48A and two arms 50 extending from the handle base. Each arm 50 defines a grip surface 50A, finger grabs 50B, and one of two teeth 50C. The grip surfaces 50A and finger grabs 50B enable the handle to be grasped or contacted by a user in order to selectively advance the catheter 42 in a distal direction during use of the insertion tool 10 to insert the catheter into the body of the patient. The teeth 50C engage corresponding raised surfaces on the hub 46 so as to removably connect the handle 48 to the catheter 42.

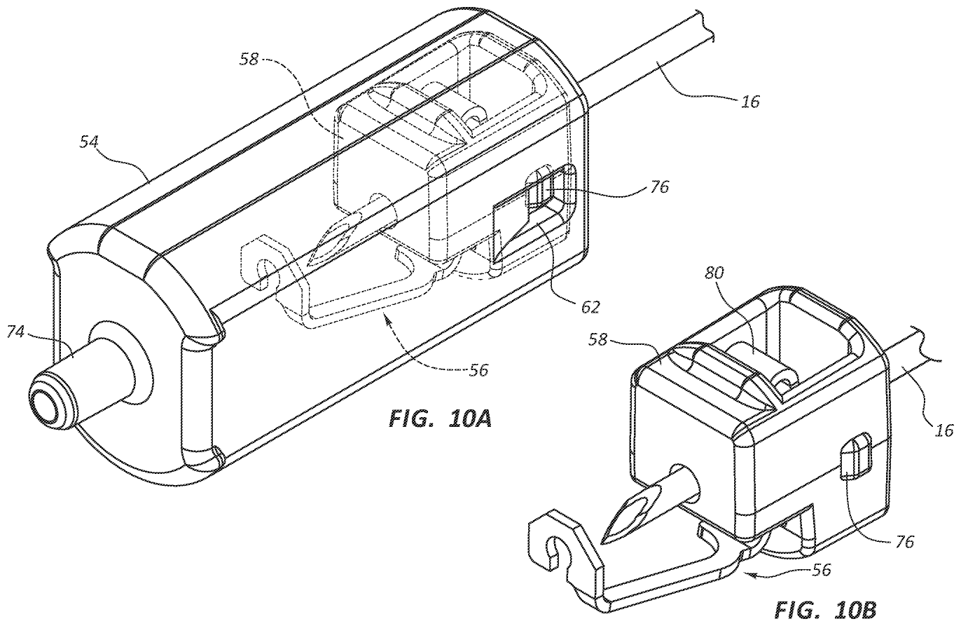

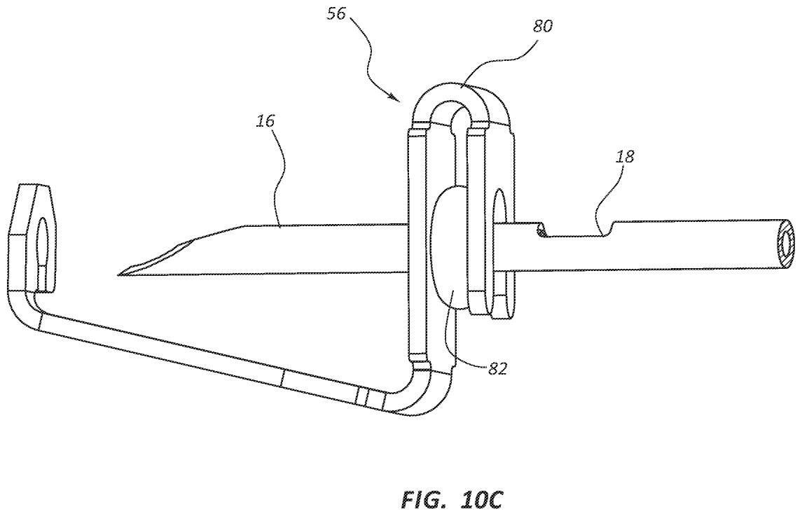

Additional components are included in relation to the handle 48 of the catheter advancement assembly 40. A plug, or valve 52, is interposed between the handle base 48A and the catheter hub 46 to prevent blood spillage when the catheter is first introduced into the patient vasculature. A safety housing 54, including a needle safety component 56 therein, is removably attached to the handle 48 between the arms 50. Specifically, protrusions 60 included on the inner surfaces of the handle arms 50 engage with corresponding recesses 62 (FIG. 10A) defined in the safety housing 54 to removably secure the safety housing to the handle 48. A cap 58 supports the needle safety component 56 and covers the end of the safety housing 54. As shown in FIG. 1B, the needle 16 initially extends through the aforementioned components in the order as shown in FIG. 2B. Further details regarding the operation of these components are given below. The cap 58, the safety housing 54, and the needle safety component 56 may combined to define a safety assembly.

Note that in one embodiment the outer diameters of the needle 16 and the catheter tube 44 are lubricated with silicone or other suitable lubricant to enhance sliding of the catheter tube with respect to the needle and for aiding in the insertion of the catheter into the body of the patient.

The insertion tool 10 further includes a support structure 70 for stabilizing the needle 16 proximate its point of exit from the housing 12. In the present embodiment, the support structure 70 includes an interface 72 of the top housing portion 12A and bottom housing 12B that is shaped to closely match the round shape of the needle 16 and catheter tube 44. The interface 72 stabilizes the needle 16 so as to prevent excessive “play” in the needle, thus improving user accuracy when initially accessing the vasculature of the patient.

As best seen in FIG. 2A, the top housing 12A, the needle hub 14, and the bottom housing 12B include engagement features 68 to maintain attachment of the proximal end of the housing 12 even when more distal portions of the housing are separated, discussed below. Note, however, that various types, sizes, and numbers of engagement features can be employed to achieve this desired functionality.

FIGS. 3A-9 depict various stages of use of the insertion tool 10 in placing the catheter 42 in the vasculature of a patient. For clarity, the various stages are depicted without actual insertion into a patient being shown. With the insertion tool 10 in the configuration shown in FIG. 1A, a user grasping the insertion tool 10 first guides the distal portion of the needle 16 through the skin at a suitable insertion site and accesses a subcutaneous vessel. Confirmation of proper vessel access having been achieved is evident via blood flash, i.e., the presence of blood between the outer diameter of the needle 16 and the inner diameter of the catheter tube 44 due to blood passing out the notch 18 from the hollow interior of the needle. Note that in one embodiment, the presence of blood in the safety housing 54, which in one embodiment is a translucent housing, can serve as a secondary blood flash indicator due to blood entering the housing from the needle 16 when the vessel is accessed.

After needle access to the vessel is confirmed, the guidewire advancement assembly 20 is actuated, wherein the slide 28 is advanced by the finger of the user to distally advance the guidewire 22 (FIGS. 3A and 3B), initially disposed within the hollow needle 16. Note that the guidewire is distally advanced by the lever 24, which is operably attached to the slide 28. Also note that during distal advancement of the slide 28, the slide arms 30 thereof travel along the rails 32 on either side of the housing 12: first the bottom housing rails 32A, then the top housing rails 32B.

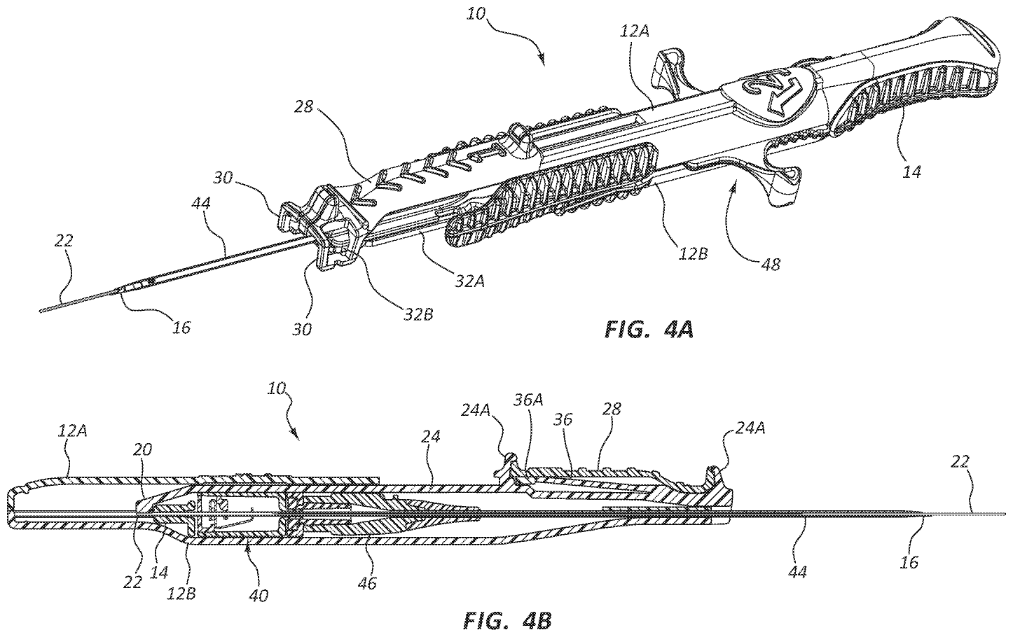

Distal guidewire advancement continues until the slide 28 has been distally slid its full travel length, resulting in a predetermined length of the guidewire 22 extending past the distal end of the needle 16, as shown in FIGS. 4A and 4B. In one embodiment, further distal advancement of the slide 28 is prevented by contact of the lever tab 26 with a distal portion of the needle hub 14, as shown in FIG. 4B. FIGS. 5A and 5B show that, upon full distal advancement of the slide 28, the slide arms 30 thereof are no longer engaged with the bottom housing rails 32A, but rather with only the top housing rails 32B. This in turn enables the housing portions 12A and 12B to separate, as seen further below.

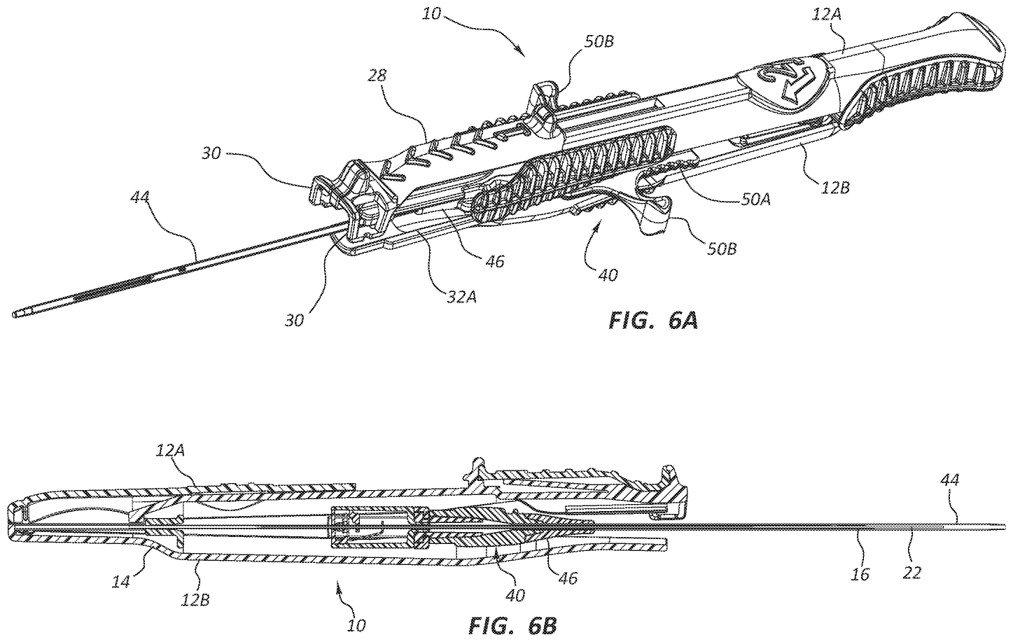

As seen in FIGS. 5A and 5B, once the guidewire 22 has been fully extended within the vessel of the patient (FIGS. 4A and 4B), the catheter advancement assembly 40 is actuated, wherein the handle 48 is distally advanced by the user to cause the catheter tube 44 to slide over distal portions of the needle 16 and guidewire 22 and into the patient's vasculature via the insertion site. FIGS. 6A and 6B show that, as the catheter is advanced via the handle 48, the housing portions 12A and 12B are easily separated so as to enable the catheter hub 46 to exit the distal end of the housing 12 and for the catheter to be inserted into the patient vasculature to a suitable degree.

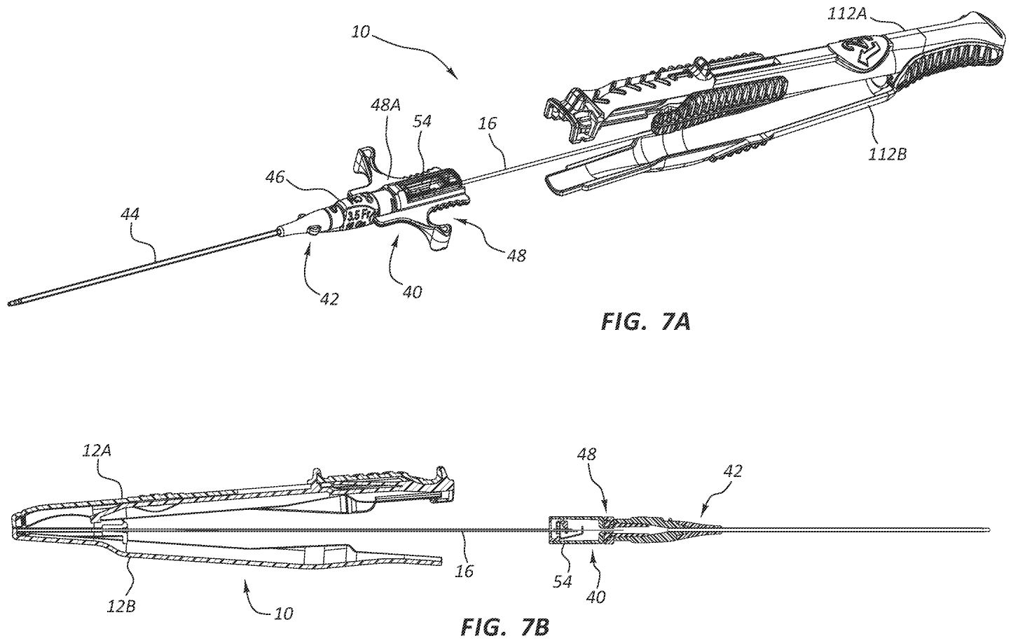

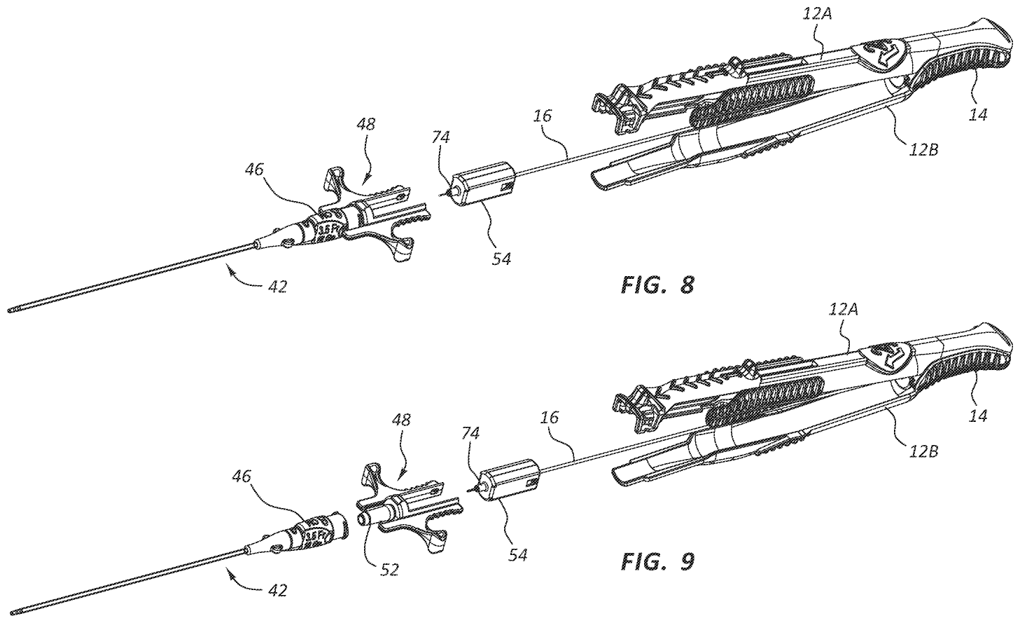

Note that, as shown in FIGS. 7A and 7B, during removal of the catheter from within the housing 12 of the insertion tool 10, the catheter slides distally along the needle 16 until the distal needle tip is received into the safety housing 54 and engaged with the needle safety component 56. FIG. 8 shows that the insertion tool 10 can then be separated from the catheter 42, leaving the handle 48 still attached to the catheter hub 46. As mentioned, the handle 48 includes the valve 52 interposed between the catheter hub 46 and the handle 48. Upon removal of the needle 16 and safety housing 54 from the catheter 42, the valve 52 occludes the catheter lumen so as to prevent inadvertent blood spillage from the catheter hub 46. As shown in FIG. 9, the handle 48 can be removed from engagement with the catheter hub 46 via pulling, twisting, etc., so as to disengage the teeth 50C of the handle from the hub. An extension leg can be attached to the catheter hub and the catheter 42 dressed down, per standard procedures. Then housing 12 and handle 48 of the insertion tool 10 can be discarded.

FIGS. 10A-10C give further details regarding the safety housing 54, as well as the needle safety component 56 and its interaction with the needle 16 in isolating the distal end thereof. As shown, the safety housing 54 is configured to enable the needle 16 to pass therethrough during use of the insertion tool 10, as has been described, exiting the housing via the extension 74 on the distal end of the housing. The cap 58 is placed into the proximal end of the safety housing 54 and is configured to support the needle safety component 56 such that he needle 16 initially passes through the safety housing, the cap, and the needle safety component. Note that the extension 74 of the safety housing 54 in the present embodiment extends into the valve 52 so as to open the valve during use of the insertion tool 10, which eliminates undesired friction between the valve and the needle.

FIG. 10C shows that the needle safety component 56 includes a bent body, or binding element 80 through which the needle initially extends, and a friction element 82. As seen in FIG. 10A, when the needle 16 is withdrawn from the catheter 42 (FIG. 8), the distal tip of the needle is withdrawn proximally through the extension 74 and past the distal portion of the needle safety component such that the needle is no longer in contact therewith. This enables the friction element 82 to cause the binding element 80 to cant slightly, thus binding the needle 16 in place and preventing its further travel with respect to the safety housing 54 and isolating the needle distal tip within the housing so as to prevent inadvertent needle sticks. In the present embodiment the friction element 82 includes a suitably sized O-ring. Suitable O-rings can be acquired from Apple Rubber Products, Lancaster, NY, for instance. Note that further details regarding the needle safety component, its operating principles, and similar devices are disclosed in U.S. Pat. Nos. 6,595,955, 6,796,962, 6,902,546, 7,179,244, 7,611,485, and 7,618,395, each of which is incorporated herein by reference in its entirety. Of course, other needle safety devices can be employed to isolate the distal end of the needle.

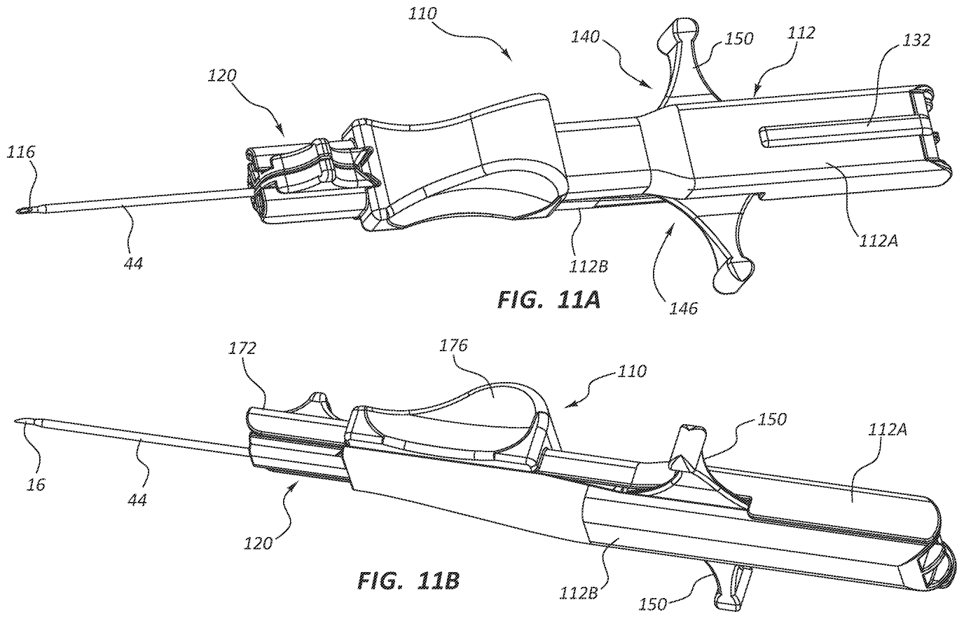

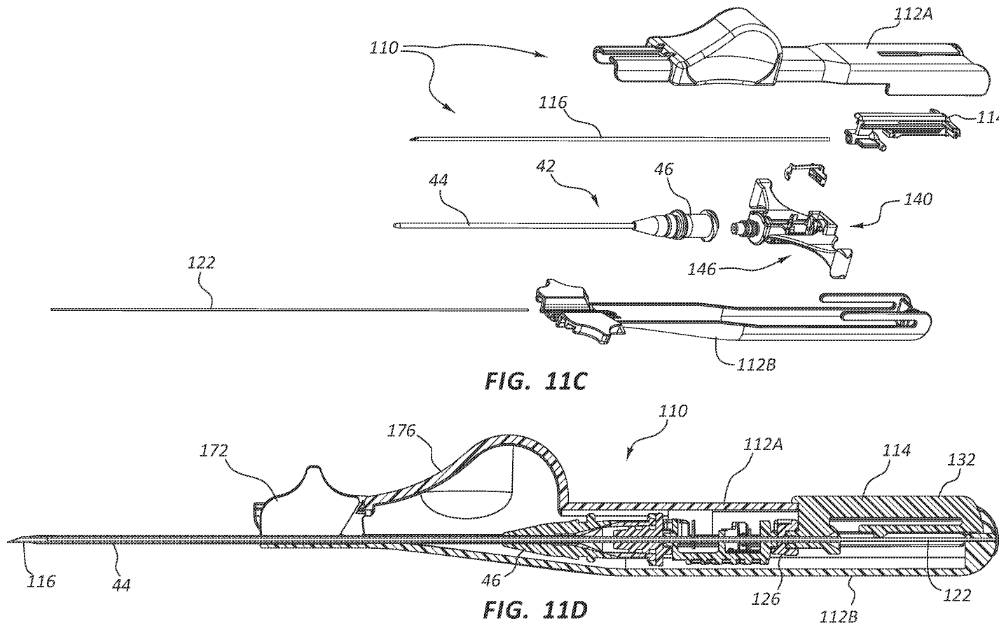

Reference is now made to FIGS. 11A-13B in describing a catheter insertion tool 110 according to one embodiment. Note that in this and succeeding embodiments, various features are similar to those already described in connection with the above embodiment. As such, only selected aspects of each embodiment to follow will be described.

The insertion tool 110 includes a housing 112 defined by a top housing portion 112A and a bottom housing portion 112B that together partially enclose the catheter 42. A needle hub 114 supporting a distally extending needle 116 is included for disposal within the housing 112 and positioned such that the catheter tube 44 of the catheter 42 is disposed over the needle. Note that partial enclosure of the catheter by the insertion tool in this and other embodiments enables a clinician to manipulate the insertion tool with hands that are closer to the distal end of the needle than what would otherwise be possible.

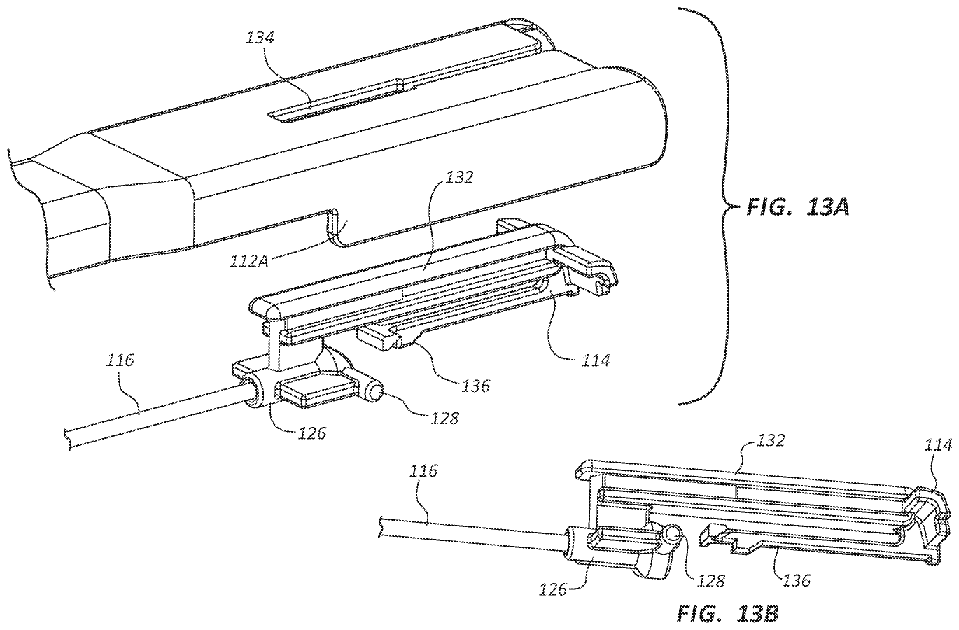

FIGS. 13A and 13B give further details regarding the needle hub 114, which is attached to the top housing portion 112A. A needle holder 126, included on a distal end of the needle hub 114, receives the proximal end of the needle 116 therein. The needle 116 is secured within the needle holder 126 via adhesive, welding, or other suitable manner. Extensions 128 are included on opposite sides of the needle holder 126 and are configured to be slidably received within corresponding slots 130 defined on the sides of the bottom housing portion 112B. Such engagement enables the bottom housing portion 112B to slide distally with respect to the top housing portion 112A.

A top rail 132 is included on the needle hub 114 and is configured to engage a corresponding slot 134 defined in the proximal portion of the top housing portion 112A so as to secure the needle hub to the top housing portion. A lock out arm 136 is also included with the needle hub 114 and positioned to engage the back plate 124 when the bottom housing portion 112B is slid distally to extend the guidewire from the needle 116, thus preventing its retraction. Note that the guidewire 122 initially distally extends from the back plate 124 and through the needle holder 126 and needle 116, as best seen in FIG. 11D.

A guidewire advancement assembly 120 is included to selectively advance a guidewire 122, initially disposed within the lumen of the needle, distally past the distal end of the needle 116. The guidewire advancement assembly 120 includes the bottom housing portion 112B to which the guidewire 122 is attached at a proximal back plate 124 thereof. As will be seen, the bottom housing portion 112B is distally slidable with respect to the top housing portion 112A to enable selective distal advancement of the guidewire 122.

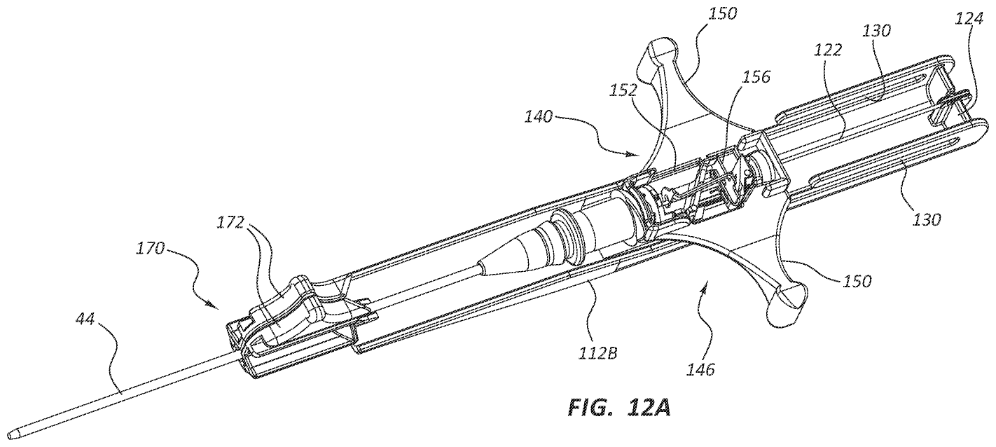

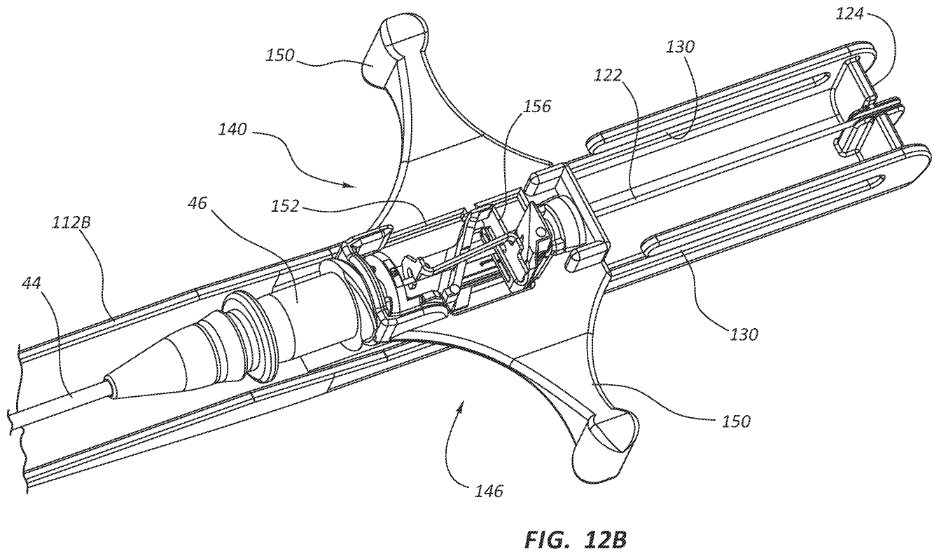

The insertion tool 110 further includes a catheter advancement assembly 140 for selectively advancing the catheter 42 over the needle 116. The advancement assembly 140 includes a handle 146 initially and slidably disposed between the top and bottom housings 112A and 112B and removably attached to the hub 46 of the catheter 42. As best seen in FIGS. 12A and 12B, the handle 146 includes two arms 150 for allowing a user to selectively slide the handle in order to advance the catheter 42. The handle 146 further includes a recess 152 in which is placed a needle safety component 156 for isolating the distal tip of the needle 116 when the needle is withdrawn from the catheter 42. Further details regarding the needle safety component are disclosed in U.S. Pat. Nos. 6,595,955, 6,796,962, 6,902,546, 7,179,244, 7,611,485, and 7,618,395, each incorporated by reference above.

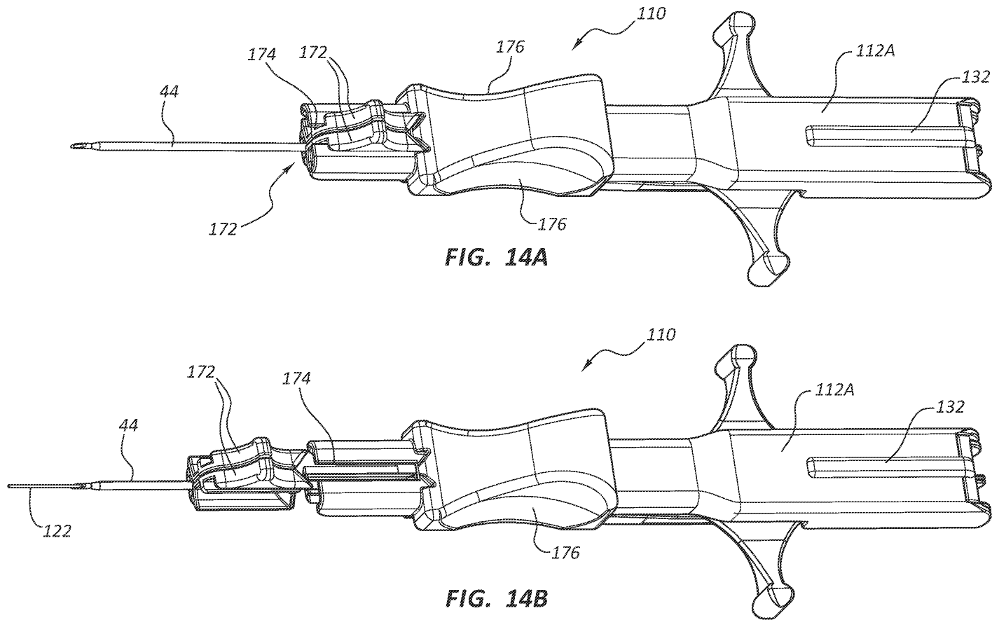

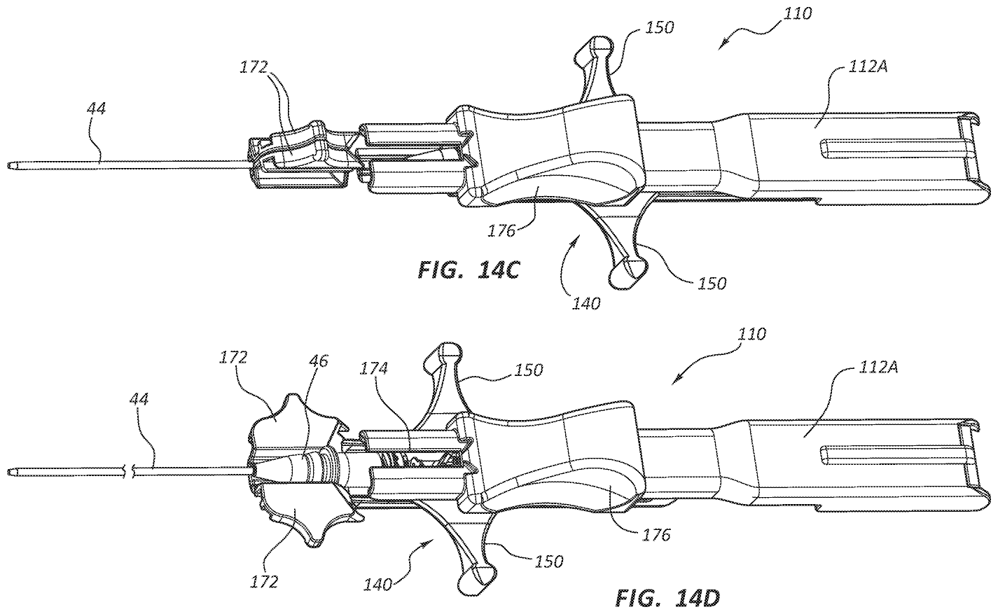

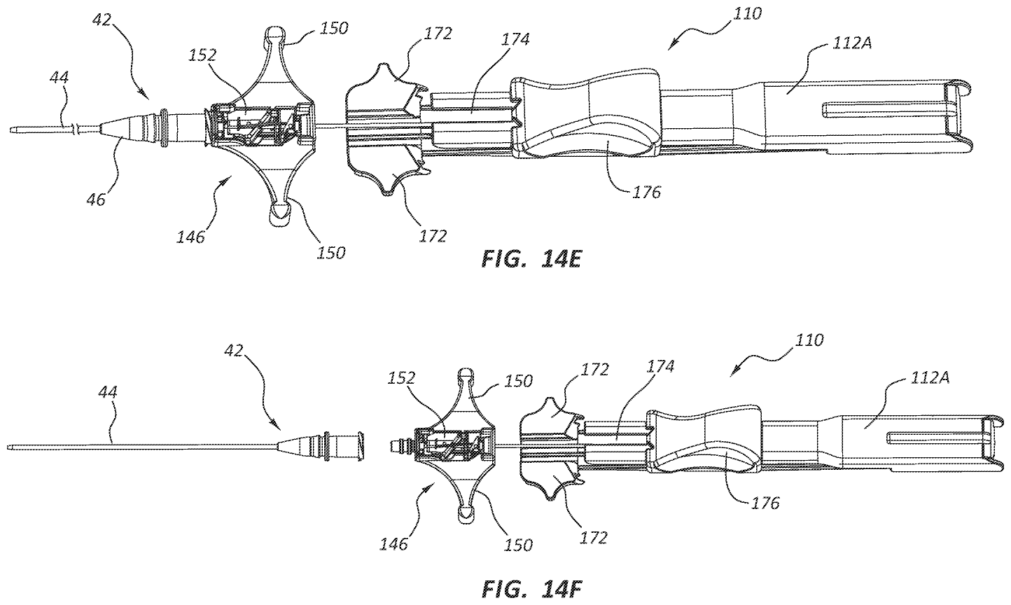

The insertion tool 110 further includes a support structure 170 for stabilizing the needle 116 proximate the distal end of the housing 112. The support structure 170 in the present embodiment includes two flaps 172 that are hingedly connected to the distal portion of the bottom housing portion 112B. When closed as seen in FIGS. 11D and 12A, the flaps 172 serve to stabilize the needle 116 to assist the user of the insertion tool 110 in inserting the needle into the patient. When open (FIG. 14D), the flaps 172 provide an opening to enable the catheter hub 46 to be removed from the distal end of the housing 112, as will be detailed further below. Before the bottom housing portion 112B is slid with respect to the top housing portion 112A, the flaps 172 are disposed in a track 174 defined by the top housing portion. Other types and configurations of support structures can also be employed. The insertion tool 110 further includes gripping surfaces 176 on either side of the housing 112 to aid in use of the tool during catheter insertion procedures, detailed below.

FIGS. 14A-14E depict various stages of use of the insertion tool 110 in inserting a catheter into a patient. With the insertion tool 110 in the configuration shown in FIG. 14A, vascular access is achieved with the needle 116 via user insertion of the needle into the patient at an insertion site. Confirmation of vessel access can be achieved via the observation of blood flashback via a distal notch in the needle 116, as described in the previous embodiment, or in other suitable ways.

Once the distal portion of the needle 116 is disposed within a vessel of the patient, the guidewire 122 is extended past the distal end of the needle and into the vessel by distally advancing the bottom housing portion 112B. Such advancement is achieved in the present embodiment by placing a user's fingers on the folded-up flaps 172 of the bottom housing portion 112B and pushing the flaps distally, thus extending the guidewire 122. The guidewire 122 is advanced until fully extended. The lock out arm 136 of the needle hub 114 then engages the back plate 124 of the bottom housing portion 112B and prevents retraction of the guidewire 122.

At this stage, the handle 146 of the catheter advancement assembly 140 is distally advanced, by a user grasping of one or both arms 150 thereof, so as to distally advance the catheter 42 through the insertion site and into the patient vasculature. This is shown in FIG. 14C, wherein the catheter tube 44 is shown distally advancing over the needle 116 and the guidewire 122.

As shown in FIG. 14D, continued distal advancement of the catheter 42 causes the catheter hub 146 to urge the flaps 172 to open, thus providing a suitable opening through which the hub may pass from the insertion tool housing 112. Note that the flaps 172 are shaped such that contact with the catheter hub 46 urges each flap to fold outward, as seen in FIG. 14D. Note also that the flaps 172 are no longer disposed within the track 174 due to full distal advancement of the guidewire 122 via finger pressure applied to the flaps 172 as described above.

FIG. 14E shows that, with the flaps no longer engaged within the track 174, the top housing portion 112A and bottom housing portion 112B are able to separate at the distal ends thereof such that the handle 146, still attached to the catheter hub 46, can separate from the housing 112. Though not shown at this stage, the needle safety component 156 disposed in the recess 152 of the handle 146 isolates the distal end of the needle 116. The handle 146 can then be manually removed from the catheter hub 46 (FIG. 14F), and placement and dressing of the catheter 42 can be completed. The insertion tool 110, including the needle 116 isolated by the needle safety component 156 of the handle 146, can be safely discarded.

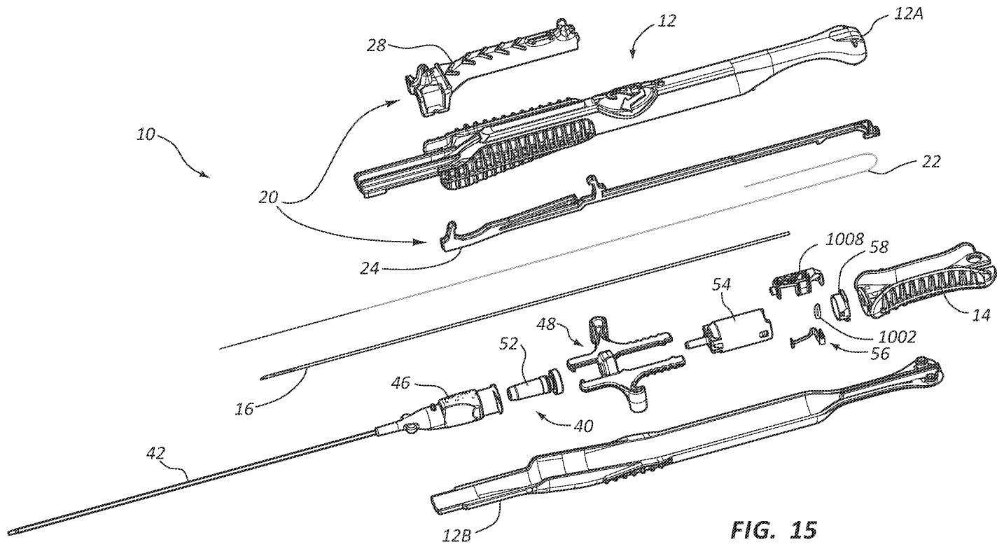

Reference is now made to FIG. 15, which depicts an exploded view of the catheter insertion device 10 according to one embodiment, including components similar to those that have already been described above. As such, only selected differences are discussed below.

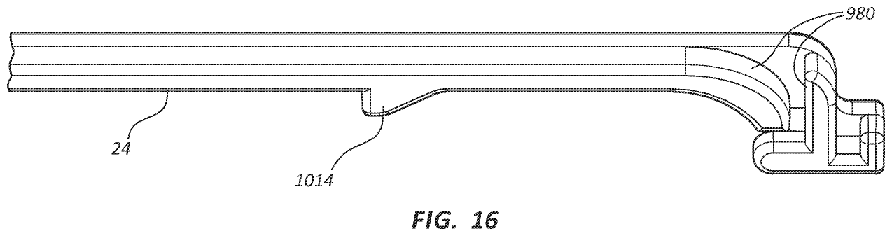

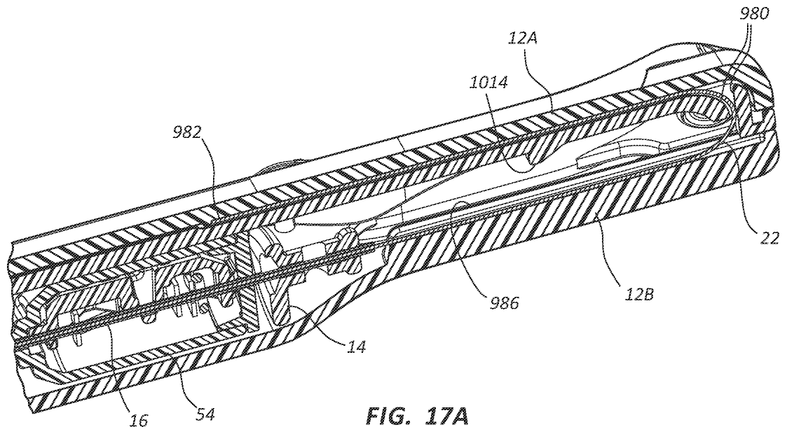

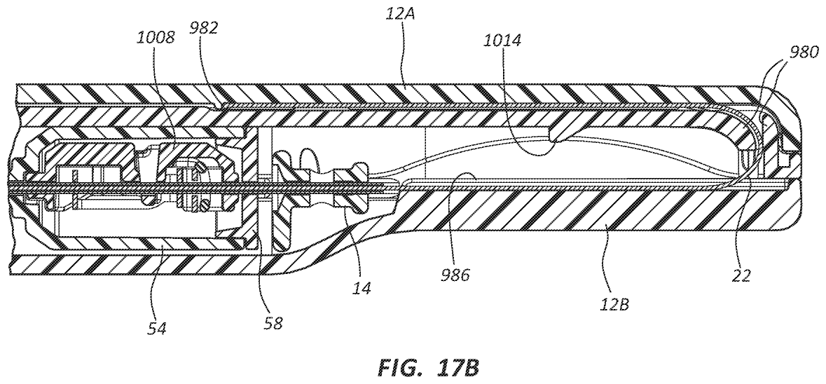

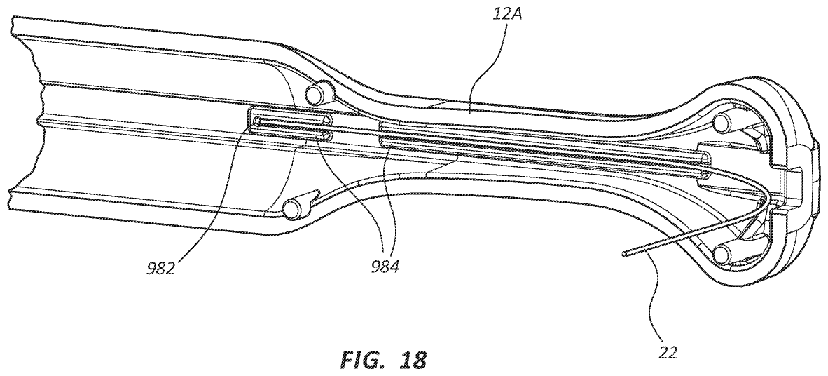

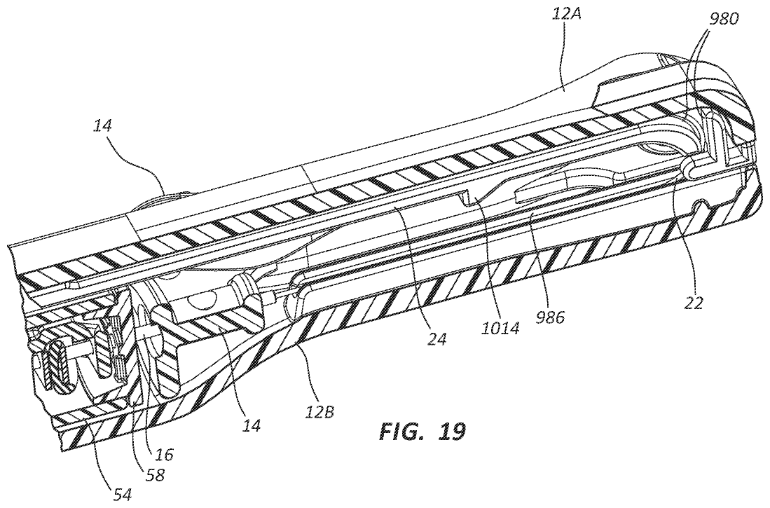

FIG. 15 shows that in the present embodiment the guidewire 22 is looped back on itself to substantially define a U-shaped configuration. FIGS. 17A and 17B show the manner in which the guidewire 22 is disposed within the housing 12 of the catheter insertion device 10. In particular, these figures show that a proximal end of the guidewire 22 is anchored to a portion of the device 10, namely, at an anchor point 982 on the top portion 12A of the housing 12. FIG. 18 shows that the guidewire 22 extends proximally and removably within a guide channel 984 defined on an interior surface of the top housing portion 12A. FIGS. 17A and 17B show that an intermediate portion of the guidewire 22 loops back on itself proximate the proximal end of the device 10. Guide surfaces 980 (FIG. 16) disposed near the proximal end of the guidewire lever 24 constrain the flexible guidewire 22 into the looped, substantially U-shaped configuration. The looped-back intermediate portion of the guidewire 22 then extends toward the distal end of the device 10 along a channel 986, best seen in FIG. 19, defined on an interior surface of the bottom housing portion 12B of the housing 12 before it passes into the hollow needle 16. The free distal end of the guidewire 22 initially resides within the needle 16.

So disposed as described immediately above, the guidewire 22 is positioned for selective advancement by the guidewire advancement assembly 20 such that the free distal end thereof can distally extend from the open distal tip of the needle 16. This selective advancement of the guidewire 22 is achieved in the present embodiment via distal movement of the guidewire advancement slide 28 included on the device housing 12. Distal movement of the guidewire advancement slide 28 causes corresponding distal sliding movement of the guidewire lever 24. The guide surfaces 980 of the guidewire lever 24 push the bend of the guidewire 22 distally as the lever advances. Note that the guidewire 22 is sufficiently rigid so as to be advanced by the guidewire lever 24 without buckling. Also, the guide surfaces 980 and guidewire 22 are configured to enable retraction of the guidewire 22 back into the insertion tool housing 12 when the guidewire advancement slide 28 or other suitable mechanism is slid proximally.

This pushing movement of the slidable guidewire lever 24 causes the distal end of the guidewire 22 to extend distally from the open distal tip of the needle 16. Because of its anchored proximal end at anchor point 982 and its bent or looped U-shape configuration, the guidewire 22 is distally advanced at a rate of about twice the rate of sliding of the guidewire advancement slide 28 and about twice the rate of guidewire advancement in the device configuration of FIGS. 1A-9, which results in about twice the length of guidewire extension when compared with the length of movement of the guidewire advancement slide 28. This further desirably results in a relatively longer length of guidewire extension into the vein or other patient vessel so as to more suitably guide the catheter 42 into the patient's body. As such, the guidewire and advancement assembly described here operates as a type of “reverse pulley” system for distal guidewire advancement. Note that other looping configurations of the guidewire can be included with the device 10 in addition to those shown and described herein. Also, differing ratios of guidewire extension vs. advancement assembly movement are also possible in other embodiments.

Note that the looping conduit and guidewire advancement handle are only examples of structures that can suitably perform the desired functionality described herein. Indeed, other structures can be employed to accomplish the principles described in connection with the present embodiment. Also, though shown and described above to be attached to the catheter insertion device housing, the proximal end of the guidewire can be attached to other structures within/on the device, such as the needle hub 14, for instance. The majority length of the guidewire in one embodiment includes a metal alloy of nickel and titanium commonly referred to as nitinol, which is sufficiently rigid and can be disposed in the U-shaped configuration without retaining a memory of that position when the guidewire is advanced. Note that other suitable guidewire materials can also be employed.

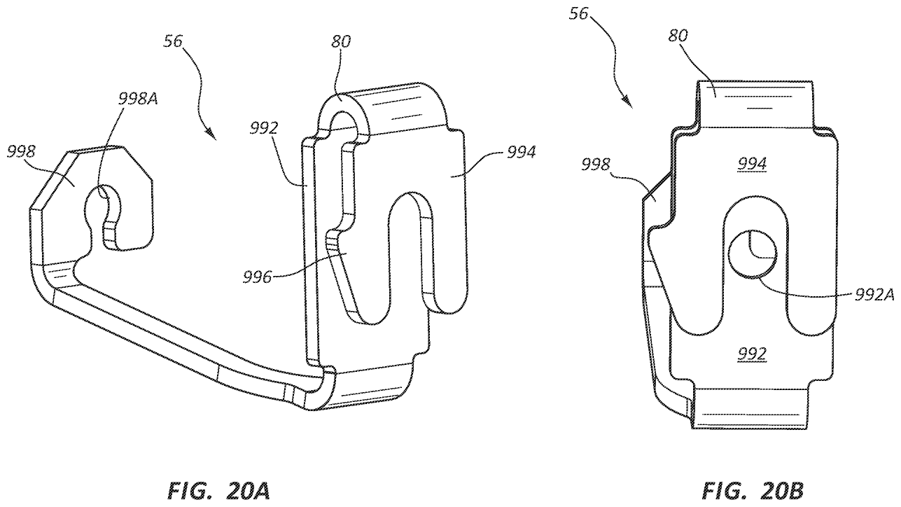

FIGS. 20A and 20B depict various details regarding the binding element 80, described further above, of the needle safety component 56 for shielding the distal tip of the needle 16 once catheter insertion is complete. As shown, the binding element 80 (which is also referred to herein as a binding member) includes a front plate 992 defining a hole 992A, and a forked back plate 994. A protuberance 996 extends from one of the forks of the back plate 994. A horseshoe-shaped needle pass-through element 998 is also included in a spaced-apart arrangement from the front plate 992 and defines a hole 998A in coaxial alignment with the hole 992A of the front plate.

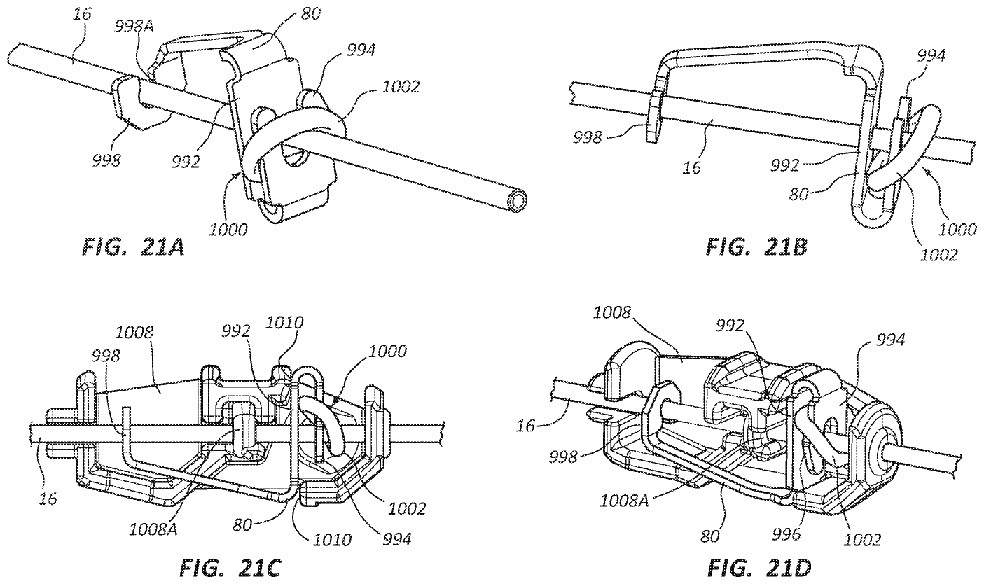

A friction element 1000, also referred to herein as a friction member, is also included with the binding element 80 in the present embodiment, namely, an annular elastomeric element, or O-ring 1002, as seen in FIGS. 21A and 21B. As shown, the O-ring 1002 is configured to wrap around both a portion of the needle 16 and the forked back plate 994. The protuberance 996 is employed to aid in maintaining the O-ring 1002 in place as shown in FIGS. 21A and 21B. With the O-ring 1002 so positioned, a relatively constant urging force is imparted by the O-ring to the binding element 80, for use in shielding the distal tip of the needle 16, as will be described further below. Note that the elastomeric element can take forms other than an O-ring while performing the same functionality. For instance, a rod or length of elastomeric material that is wrapped about a portion of the binding element and the needle could also be employed.

FIGS. 21C and 21D show the binding element 80 disposed in the carriage 1008, which is in turn disposed within the safety housing 54. As shown, the carriage 1008 defines two constraining surfaces 1010 against which corresponding portions of the front plate 992 of the binding element initially rest when the needle 16 initially extends through the carriage and the binding element. A retaining ring 1008A through which the needle 16 slidably passes enables engagement of the needle with the carriage 1008.

The binding element 80 is initially slidably disposed with the needle 16 in the state shown in 21A-21D (showing the binding element before it has shielded the distal tip of the needle) such that relative sliding movement between the needle and the binding element is permitted. Passage of the needle 16 through the hole 998A of the needle pass-through element 998 initially limits canting movement of the binding element 80.

The needle 16 also passes through the hole 992A of the front plate 992 such that the needle is straddled by the forks of the forked back plate 994. As mentioned, the O-ring 1002 is disposed about the needle 16 and the back plate 994 so as to provide a drag force when the carriage 1008 and binding element 80 (both housed within the safety housing 54 (FIG. 15) are slid distally along the length of the needle 16 during use of the device 10. The drag force provided by the O-ring 1002 during such distal sliding in turn imparts a rotational moment on the binding element 80 (by virtue of forces provided via the contact of the binding element with the O-ring) to urge the binding element to rotate in a clockwise motion, from the perspective of the drawing shown in FIG. 21C.

Such clockwise rotation of the binding element 80 is prevented by the needle pass-through feature 998 while the needle 16 extends through the binding element. Once the safety housing 54 containing the carriage 1008 and binding element 80 has been slid distally a sufficient distance such that the needle pass-through element 998 slides past and off the distal end of the needle 16, however, the binding element is no longer constrained and the drag force imparted by the O-ring 1002 causes the binding element to cant clockwise with respect to the needle, from the perspective of the drawing shown in FIG. 21C. This canting locks movement of the binding element 80 and, by extension, the carriage 1008, with respect to the needle 16, by virtue of physical binding between the outer surface of the needle 16 with the perimeter of the front plate hole 992A, which thus acts as a binding surface. With the distal tip of the needle 16 safely disposed within the locked carriage 1008, the user is thus protected from an accidental needle stick.

As mentioned above, the O-ring 1002 imparts a relatively constant urging force for canting the binding element 80, which keeps the binding element canted (after withdrawal of the needle distal tip into the carriage as described above) so as to more securely lock the carriage 1008 over the distal tip of the needle 16. This constant urging force is beneficial, for example, in instances when the needle 16 is pushed back and forth with respect to safety housing 54/carriage 1008 after it has been locked over the needle distal tip to ensure that the binding element does not return to an orientation in which the needle pass-through feature 998 can re-engage the needle 16 and unlock the needle safety component 56. Note that the O-ring 1002 can be employed with needles and binding elements larger or smaller than those shown and described herein.

The O-ring 1002 in the above embodiments is sufficiently compliant so as to stretch over the aforementioned structures while imparting the desired force, as explained above. In one embodiment, the O-ring 1002 material includes any one or more of natural or synthetic rubber, elastomers, polymers, thermoplastics, silicones, etc. In one embodiment, the O-ring material is selected so as to provide sufficient tear resistance, ability to impart the desired friction, and chemical compatibility. The size of the O-ring can vary according to the size and configuration of the binding element and needle. In other embodiments, the O-ring can include other shapes, materials, and positional placements while still providing the intended functionality.

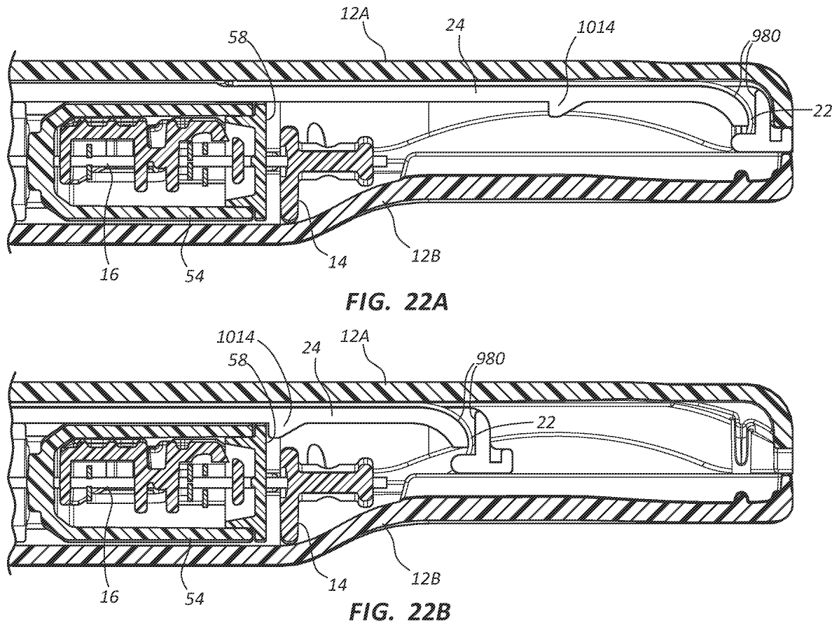

FIG. 22A shows that the guidewire lever 24 can include a catheter advancement feature that enables the guidewire lever to distally advance the catheter 42 in addition to advancing the guidewire 22 as described above. In the present embodiment, the catheter advancement feature includes an advancement tab 1014 disposed on the proximal portion 24A of the guidewire lever 24 and disposed so as to physically engage the cap 58 of the safety housing 54 when the guidewire lever 24 is moved distally via distal sliding by the user of the slide 28 (FIG. 15). Such engagement is shown in FIG. 22B. Further distal movement of the guidewire lever 24 results in distal advancement of the safety can 54 and the catheter 42 indirectly but operably attached thereto (FIG. 15). The slide 28 in the present embodiment can be slid to distally advance the catheter 42 a predetermined distance via the advancement tab 1014 of the guidewire lever 24. In one embodiment, the predetermined distance advances the catheter 42 until its distal end distally advances over the distal tip of the needle 16. Further distal advancement of the catheter 42 can be achieved via distal sliding of the handle 48 as needed (FIG. 15). In another embodiment, the slide 28 is configured to distally advance the catheter the full distal distance needed, via the advancement tab 1014.

The position of the advancement tab 1014 of FIG. 22A is such so as to provide staged advancement of the guidewire 22 and catheter 42. In particular, distal advancement of the guidewire lever 24 from the position shown in FIG. 22A produces immediate advancement of the guidewire 22 while the safety housing 54 and catheter 42 remain in place. Further distal advancement of the guidewire lever 24 to the position shown in FIG. 22B causes the advancement tab 1014 to engage and distally advance the safety can 54 and catheter 42, as described above, while continuing to distally advance the guidewire 22.

Thus, in addition to distally advancing the guidewire 22 out through the needle 16, the guidewire lever 24 can also advance the catheter 42 distally along the needle 16 and into a vessel of the patient, as described further above. Note that the particular shape and configuration of the advancement tab 1014, together with its manner of engagement with, and magnitude of travel imparted to, the safety housing and/or catheter can vary from what is shown and described herein.

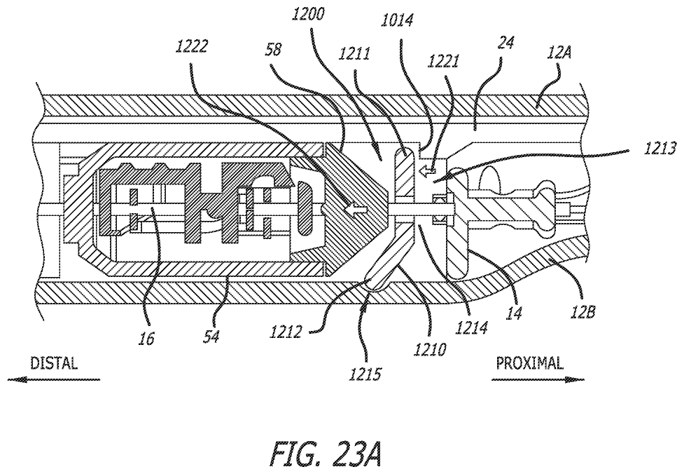

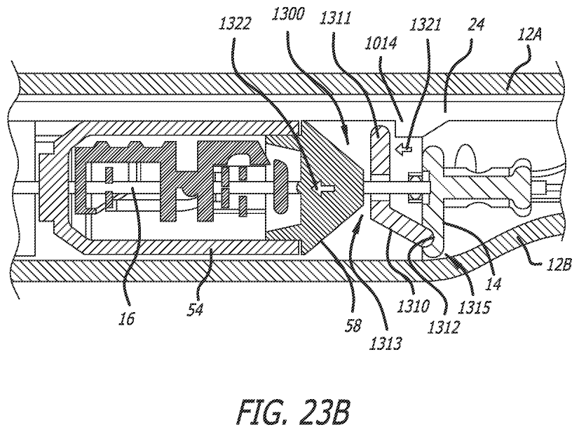

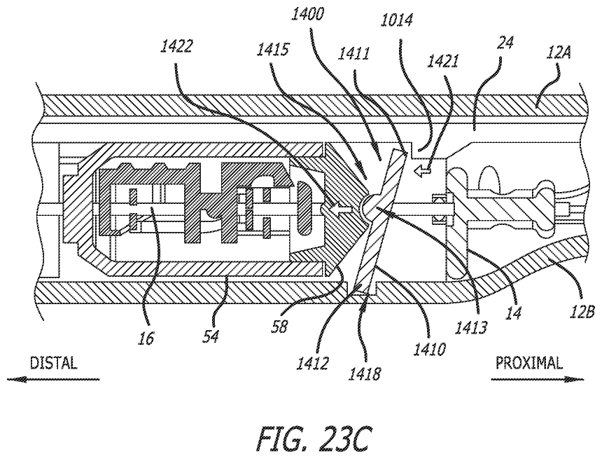

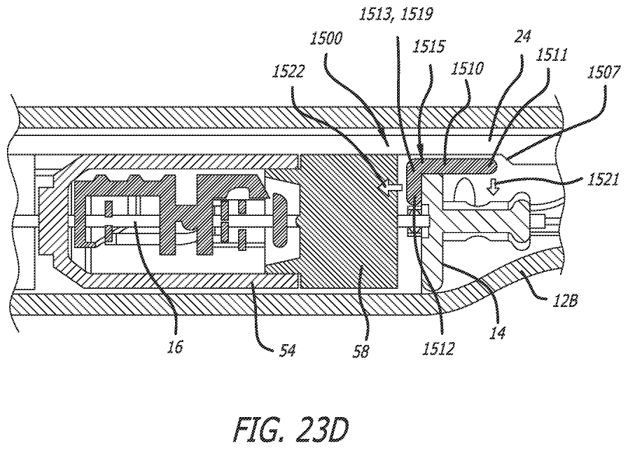

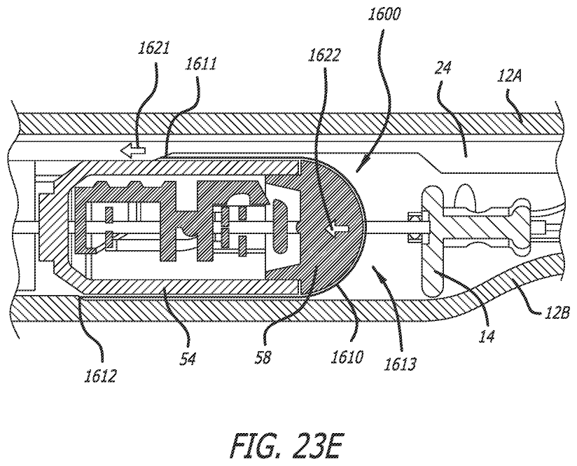

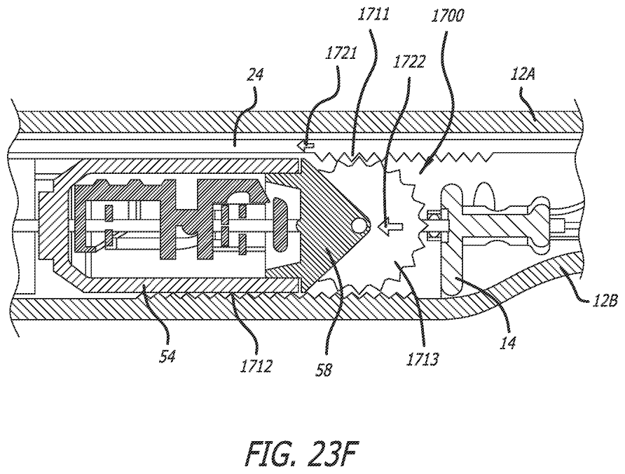

FIG. 23A illustrates a detailed cross-sectional side view of a portion of the insertion tool 10 (sometimes referred to a catheter insertion device assembly) incorporating a first embodiment of a mechanical advantage mechanism, according to some embodiments. The mechanical advantage mechanism 1200 is generally configured to increase a force applied to the catheter 42 by the slide 28 (FIGS. 1A, 1B) or more specifically, the guidewire lever 24 of the slide 28. More specifically, the guidewire lever 24 applies an input force 1221 to the mechanical advantage mechanism 1200 and in turn, the mechanical advantage mechanism 1200 applies an output force 1222 to the cap 58 of the safety housing 54. The cap 58, the safety housing 54, the catheter hub 46, and the catheter 42 are coupled together such that the cap 58, the safety housing 54, the catheter hub and the catheter 42 displace distally with respect to the bottom housing portion 12B as a single unit, and such that a distally oriented force applied to the cap 58 is transferred to the catheter 42 (FIG. 15). In use, the user applies the input force 1221 to the slide 28 and the slide 28 transfers the input force 1221 to the mechanical advantage mechanism 1200 via the advancement tab 1014 of the guidewire lever 24. As a result, the mechanical advantage mechanism 1200 applies the output for 1222 to the catheter 42 to distally displace the catheter 42 along the needle 16. The mechanical advantage mechanism 1200 is generally configured such that the output force 1222 is greater than the input force 1221. In some embodiments, the output force 1222 may be twice as much or more as the input force 1221.

The mechanical advantage mechanism 1200 includes a lever 1210. The lever 1210 defines a first end 1211, a second end 1212 and an intermediate point 1213 between the first end 1211 and the second end 1212. The second end 1212 defines a fulcrum 1215 with the bottom housing portion 12B. In some embodiments, the lever 1210 may include an opening 1214 configured to accommodate passage of the needle 16 therethrough. The opening 1214 may be include a hole or a slot.

The first end 1211 is coupled with the guidewire lever 24 via the advancement tab 1014 such that a distal displacement of the slide 28 causes a corresponding distal displacement of the first end 1211. Said another way, the advancement tab 1014 transfers the input force 1221 to the first end 1211 causing the first end 1211 to distally displace with respect to the bottom housing portion 12B which in turn causes the lever 1210 to rotate about the fulcrum 1215. The rotation of the lever 1210 causes the intermediate point 1213 to contact the cap 58 such that the intermediate point 1213 applies the output force 1222 to the cap 58.

FIG. 23B illustrates a detailed cross-sectional side view of a portion of the insertion tool 10 incorporating a second embodiment of a mechanical advantage mechanism, according to some embodiments. The mechanical advantage mechanism 1300 is generally configured to increase a force applied to the catheter 42 by the guidewire lever 24. More specifically, the guidewire lever 24 applies an input force 1321 to the mechanical advantage mechanism 1300 and in turn, the mechanical advantage mechanism 1300 applies an output force 1322 to the cap 58 of the safety housing 54. The cap 58, the safety housing 54, the catheter hub 46, and the catheter 42 are coupled together such that the cap 58, the safety housing 54, the catheter hub and the catheter 42 displace distally with respect to the bottom housing portion 12B as a single unit, and such that a distally oriented force applied to the cap 58 is transferred to the catheter 42. In use, the user applies the input force 1321 to the slide 28 (FIGS. 1A, 1B) and guidewire lever 24 of the slide 28 transfers the input force 1321 to the mechanical advantage mechanism 1300. As a result, the mechanical advantage mechanism 1300 applies the output for 1322 to the catheter 42 to distally displace the catheter 42 along the needle 16. The mechanical advantage mechanism 1300 is generally configured such that the output force 1322 is greater than the input force 1321. In some embodiments, the output force 1322 may be twice as much or more as the input force 1321.

The mechanical advantage mechanism 1300 includes a lever 1310. The lever 1310 defines a first end 1311, a second end 1312 and an intermediate point 1313 between the first end 1311 and the second end 1312. The second end 1312 defines a fulcrum 1315 with the needle hub 14. The first end 1311 is coupled with the guidewire lever 24 via the advancement tab 1014 such that a distal displacement of the slide 28 causes a corresponding distal displacement of the first end 1311. Said another way, the advancement tab 1014 transfers the input force 1321 to the first end 1311 causing the first end 1311 to distally displace with respect to the bottom housing portion 12B which in turn causes the lever 1310 to rotate about the fulcrum 1315. The rotation of the lever 1310 causes the intermediate point 1313 to contact the cap 58 such that the intermediate point 1313 applies the output force 1322 to the cap 58.

FIG. 23C illustrates a detailed cross-sectional side view of a portion of the insertion tool 10 (sometimes referred to a catheter insertion device assembly) incorporating a third embodiment of the mechanical advantage mechanism, according to some embodiments. The mechanical advantage mechanism 1400 may in some respects resemble the features and functionalities of the mechanical advantage mechanisms described above. The mechanical advantage mechanism 1400 includes a lever 1410. The lever 1410 defines a first end 1411, a second end 1412 and an intermediate point 1413 between the first end 1411 and the second end 1412. The second end 1412 engages with the bottom housing portion 12B via an engagement feature 1418 so that distal displacement of the second end 1412 with respect to bottom housing portion 12B is limited or prevented. The engagement feature 1418 may include a tab, a slot, or any other suitable feature. The intermediate point 1413 defines a fulcrum with the cap 58. The first end 1411 is coupled with the guidewire lever 24 via the advancement tab 1014 such that a distal displacement of the slide 28 (FIGS. 1A, 1B) causes a corresponding distal displacement of the first end 1411. Said another way, the advancement tab 1014 transfers the input force 1421 to the first end 1411 causing the first end 1411 to distally displace with respect to the bottom housing portion 12B which in turn causes the lever 1410 to rotate about the fulcrum 1415. The rotation of the lever 1410 causes the intermediate point 1413 to contact the cap 58 such that the intermediate point 1413 applies the output force 1422 to the cap 58.

FIG. 23D illustrates a detailed cross-sectional side view of a portion of the insertion tool 10 (sometimes referred to a catheter insertion device assembly) incorporating a fourth embodiment of the mechanical advantage mechanism, according to some embodiments. The mechanical advantage mechanism 1500 may, in some respects, resemble the features and functionalities of the mechanical advantage mechanisms described above. The mechanical advantage mechanism 1500 includes a lever 1510. The lever 1510 defines a first end 1511, a second end 1512 and a bend 1519 at an intermediate point 1513 between the first end 1511 and the second end 1512. The bend 1519 defines a horizontal section of the lever 1510 extending between the intermediate point 1513 and the first end 1511 and a vertical section extending between the intermediate point 1513 and the second end 1512. The second end 1512 engages the cap 58 so that distal displacement of the second end 1512 causes distal displacement of the cap 58. The intermediate point 1513 defines a fulcrum 1515 with the needle hub 14. The first end 1511 is slidably coupled with the guidewire lever 24 so that a cam surface 1507 of the guidewire lever 24 causes a transverse inward/downward displacement of the first end 1511. The inward/downward displacement of the first end 1511 in turn causes distal displacement of the second end 1512. Said another way, the cam surface 1507 transfers the input force 1521 to the first end 1511 causing the first end 1511 to displace toward the bottom housing portion 12B which in turn causes the lever 1510 to rotate about the fulcrum 1515. The rotation of the lever 1510 causes the second end 1512 to contact the cap 58 such that the second end 1512 applies the output force 1522 to the cap 58. As such, rotation of the lever 1510 separation of the cap 58 from the needle hub 14.

FIG. 23E illustrates a detailed cross-sectional side view of a portion of the insertion tool 10 (sometimes referred to a catheter insertion device assembly) incorporating a fifth embodiment of the mechanical advantage mechanism, according to some embodiments. The mechanical advantage mechanism 1600 may, in some respects, resemble the features and functionalities of the mechanical advantage mechanisms described above. The mechanical advantage mechanism 1600 includes a tension member 1610 (e.g., a wire or a cable). The tension member 1610 defines a first end 1611, a second end 1612 and a loop portion 1613 between the first end 1611 and the second end 1612. The tension member 1610 is attached to the guidewire lever 24 of the slide 28 (FIGS. 1A, 1B) at the first end 1611. The tension member 1610 extends proximally away from the first end 1611 along the guidewire lever 24 between the guidewire lever 24 and the safety housing 54. The loop portion 1613 wraps partially around the cap 58 and then extends distally along the bottom housing portion 12B between the safety housing 54 and the bottom housing portion 12B. The second end 1612 is attached to the bottom housing portion 12B. Distal displacement of the slide 28 pulls the loop portion 1613 distally such that the tension member 1610 slides along the cap 58. The distal displacement of the loop portion 1613 pulls the cap 58 distally along therewith such that the distal displacement of the cap 58 is one-half the distal displacement of the slide 28. Said another way, the guidewire lever 24 exerts an input force 1621 on the tension member 1610 at the first end 1611 and the loop portion 1613 exerts an output force on the cap 58, where the output force 1622 is twice as much as the input force 1621. The cap 58 is coupled with the catheter 42 such that distal displacement of the cap causes a corresponding distal displacement of the catheter 42 with respect to the bottom housing portion 12B and the needle 16. In some embodiments, the tension member 1610 may include some slack (i.e., may not be taught) when the slide 28 is in a fully retracted position, i.e., fully displaced to the right in FIG. 23E. In such embodiments, the guidewire lever 24 may not cause the cap 58 to displace until the guidewire 22 is inserted a defined distance.

FIG. 23F illustrates a detailed cross-sectional side view of a portion of the insertion tool 10 (sometimes referred to a catheter insertion device assembly) incorporating a sixth embodiment of the mechanical advantage mechanism, according to some embodiments. The mechanical advantage mechanism 1700 may, in some respects, resemble the features and functionalities of the mechanical advantage mechanisms described above. The mechanical advantage mechanism 1700 includes a first gear rack 1711, a second gear rack 1712, and a pinion gear 1713 disposed between the first gear rack 1711 and the second gear rack 1712 such that the pinion gear 1713 is in mesh with both the first gear rack 1711 and the second gear rack 1712. The first gear rack 1711 is coupled with (e.g., attached to or incorporated within) the slide 28 (FIGS. 1A, 1B) or more specifically the guidewire lever 24 and the second gear rack 1712 is coupled with (e.g., attached to or incorporated within) the bottom housing portion 12B. Distal displacement of the slide 28 causes the pinon gear 1713 to roll along (i.e., rotate and distally displace along) the second gear rack 1712. The pinion gear 1713 is coupled with the catheter 42 via the cap 58 such that the pinion gear 1713 and the catheter 42 distally displace as a single unit. The rolling of the pinion gear 1713 along the second gear rack 1712 causes a distal displacement of the pinion gear 1713 that is one-half the distal displacement of the slide 28. Said another way, the slide 28 exerts an input force 1721 on the circumference (or top) of the pinion gear 1713 and the center of the pinion gear 1713 exerts an output force on the cap 58, where the output force 1722 is twice as much as the input force 1721. The cap 58 is coupled with the catheter 42 such that distal displacement of the cap 58 causes a corresponding distal displacement of the catheter 42 with respect to the bottom housing portion 12B and the needle 16.

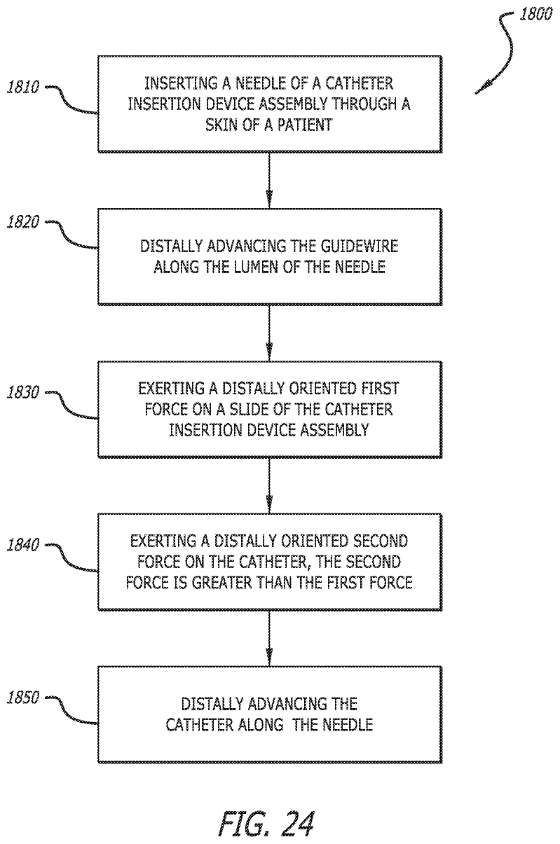

FIG. 24 is a block diagram of a method of placing a catheter within a blood vessel that, according to some embodiments, may include all or any subset of the following steps, actions, or processes. The method 1800 may include inserting a needle of a catheter insertion device assembly through the skin of a patient (block 1810) such that a distal end of the needle is disposed within the blood vessel. The needle is pre-disposed within a lumen of a catheter of the catheter insertion device assembly, and a guidewire of the catheter insertion device assembly is pre-disposed with a lumen of the needle. The method 1800 may further include distally advancing the guidewire along the lumen of the needle (block 1820) such that the guidewire extends beyond the distal end of the needle.

The method 1800 may further include exerting a distally oriented first force on the slide (block 1830). The method 1800 may further include exerting a distally oriented second force on the catheter (block 1840), where the second force is greater than the first force.

The method 1800 may further include distally advancing the catheter a catheter distance along the needle (block 1850) such that a distal end of the catheter is displaced from a position proximal the distal end the needle to a position distal the distal end the needle. In some embodiments of the method 1800, distally advancing the catheter includes distally displacing a slide of the catheter insertion device assembly a slide distance with respect to a housing of the catheter insertion device assembly, and where the slide distance is greater than the catheter distance.

In some embodiments of the method 1800, distally displacing the slide includes rotating a lever about a fulcrum, where the fulcrum coupled with a hub of the catheter, a hub of the needle, or the housing.

In some embodiments of the method 1800, the catheter insertion device includes a tension member having (i) a first end coupled with the slide, (ii) a second end coupled with the housing, and (iii) a loop portion coupled with the catheter. In such embodiments, a distal displacement of the slide with respect to the housing causes the loop portion to distally displace the catheter along the needle.

In some embodiments of the method 1800, the catheter insertion device includes (i) a first gear rack coupled with the slide, (ii) a second gear rack coupled with the housing, and (iii) a pinion gear coupled with the catheter, where the pinion gear in mesh with the first gear rack and the second gear rack. In such embodiments, a distal displacement of the slide with respect to the housing causes the pinion gear to rotate and distally displace along the housing thereby causing the catheter to distally displace along the needle.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A catheter insertion device assembly, comprising:
a catheter including a catheter tube defining a catheter lumen extending between a catheter distal end and a catheter hub at a catheter proximal end, the catheter hub disposed within a housing;
a needle configured for insertion into a patient between a skin surface and a blood vessel, the needle defining a needle lumen extending between a needle distal end and a needle proximal end coupled with the housing, wherein the needle is pre-disposed within the catheter lumen such that the needle distal end extends beyond the catheter distal end and the needle proximal end extends proximally beyond the catheter hub;
a guidewire extending between a guidewire distal end and a guidewire proximal portion, wherein the guidewire is pre-disposed within the needle lumen such that the guidewire distal end is positioned proximal the needle distal end and the guidewire proximal portion extends proximally beyond the needle proximal end; and
a slide displaceable along an exterior of the housing, the slide coupled with the guidewire proximal portion such that a displacement of the slide causes a displacement of the guidewire; and
a mechanical advantage mechanism coupled between the slide and the catheter hub such that:
the slide provides an input force to the mechanical advantage mechanism, and
the mechanical advantage mechanism provides an output force to the catheter hub in response to the input force, the output force greater than the input force.

2. The assembly according to claim 1, wherein the input force and the output force are each distally oriented.

3. The assembly according to claim 1, wherein the output force is greater than the input force by a factor of two.

4. The assembly according to claim 1, wherein the displacement of the slide causes a displacement of the catheter and a simultaneous displacement of the guidewire.

5. The assembly according to claim 4, wherein the displacement of the catheter is less than the simultaneous displacement of the guidewire.

6. The assembly according to claim 1, wherein the mechanical advantage mechanism includes a lever.

7. The assembly according to claim 6, wherein the lever includes an opening and the needle passes through the opening.

8. The assembly according to claim 6, wherein:
the lever includes:
a first end coupled with the slide;
a second end defining a fulcrum with a bottom housing portion; and
an intermediate point coupled with the catheter hub, and
a distal displacement of the slide with respect to the bottom housing portion causes a distal displacement of the catheter with respect to the needle.

9. The assembly according to claim 6, wherein:
the lever includes:
a first end coupled with the slide;
a second end defining a fulcrum with a hub of the needle; and
an intermediate point coupled with the catheter hub, and
a distal displacement of the slide with respect to the bottom housing portion causes a distal displacement of the catheter with respect to the needle.

10. The assembly according to claim 6, wherein:
the lever includes:
a first end coupled with the slide;
a second end coupled with a hub of the needle; and
an intermediate point coupled with the catheter hub, the intermediate point defining a fulcrum, and
a distal displacement of the slide with respect to the bottom housing portion causes a distal displacement of the catheter with respect to the needle.

11. The assembly according to claim 6, wherein:
the lever includes:
a first end slidably coupled with a cam surface of the slide;
a second end coupled with the catheter hub; and
intermediate point adjacent a bend of the lever defining a fulcrum with a hub of the needle, and
a distal displacement of the slide with respect to the bottom housing portion causes a transverse displacement of the first end which in turn causes a distal displacement of the catheter with respect to the needle.

12. The assembly according to claim 1, wherein:
the mechanical advantage mechanism includes a tension member having:
a first end coupled with the slide;
a second end coupled with a bottom housing portion; and
a loop portion coupled with the catheter hub, and
a distal displacement of the slide with respect to the bottom housing portion causes the loop portion to distally displace the catheter with respect to the needle.

13. The assembly according to claim 1, wherein:
the mechanical advantage mechanism includes:
a first gear rack coupled with the slide;
a second gear rack coupled with a bottom housing portion; and
a pinion gear coupled with the catheter hub, the pinion gear in mesh with the first gear rack and the second gear rack, and
a distal displacement of the slide with respect to the bottom housing portion causes the pinion gear to rotate and distally displace along the bottom housing portion resulting in distal co-displacement of the catheter with the pinion gear with respect to the needle.

14. The assembly according to claim 1, further including a safety assembly configured to cover a distal tip of the needle upon withdrawal of the needle from the catheter, the safety assembly coupled between the catheter hub and the mechanical advantage mechanism such that distal displacement of the slide causes distal displacement of the safety assembly which in turn causes distal displacement of the catheter.

15. The assembly according to claim 1, wherein:
the slide is configured to displace a first distance and a subsequent second distance,
displacement of the slide the first distance causes the guidewire to distally displace a first guidewire distance with respect to the needle, and
displacement of the slide the subsequent second distance causes:
the guidewire to distally displace a second guidewire distance with respect to the needle, and
the catheter to displace a first catheter distance with respect to the needle, the first catheter distance less than the second guidewire distance.

16. A method of placing a catheter within a blood vessel, comprising:
inserting a needle of a catheter insertion device assembly through a skin of a patient such at a distal end of the needle is disposed within the blood vessel, wherein:
the needle is pre-disposed within a lumen of a catheter of the catheter insertion device assembly, and
a guidewire of the catheter insertion device assembly is pre-disposed with a lumen of the needle;
distally advancing the guidewire along the lumen of the needle such that the guidewire extends beyond the distal end of the needle; and
distally advancing the catheter a catheter distance along the needle such that a distal end of the catheter is displaced from a position proximal the distal end the needle to a position distal the distal end the needle,
wherein:
distally advancing the catheter includes distally displacing a slide of the catheter insertion device assembly a slide distance with respect to a housing of the catheter insertion device assembly, and
the slide distance is greater than the catheter distance.

17. The method according to claim 16, wherein:
distally displacing the slide includes exerting a distally oriented first force on the slide,
distally advancing the catheter includes exerting a distally oriented second force on the catheter, and
the second force is greater than the first force.

18. The method according to claim 16, wherein distally displacing the slide includes rotating a lever about a fulcrum, the fulcrum coupled with a hub of the catheter, a hub of the needle or the housing.

19. The method according to claim 16, wherein:
the catheter insertion device includes a tension member having:
a first end coupled with the slide;
a second end coupled with the housing; and
a loop portion coupled with the catheter, and
a distal displacement of the slide with respect to the housing causes the loop portion to distally displace the catheter along the needle.

20. The method according to claim 1, wherein:
the catheter insertion device includes:
a first gear rack coupled with the slide;
a second gear rack coupled with the housing; and
a pinion gear coupled with the catheter, the pinion gear in mesh with the first gear rack and the second gear rack, and
a distal displacement of the slide with respect to the housing causes the pinion gear to rotate and distally displace along the housing thereby causing the catheter to distally displace along the needle.
